(19) United States
(12) Patent Application Publication
(10) Pub. No.: US 2025/0366894 A1
(43) Pub. Date: Dec. 4, 2025

(54) FUNCTIONAL GRADIENT LATTICE IN ORTHOPEDIC FIXATION IMPLANT

(71) Applicant: Lattice Orthopedics, LLC, Newport Coast, CA (US)

(72) Inventors: Jay Levin, Durham, NC (US); Michael Brown, Torrance, CA (US)

(21) Appl. No.: 19/220,997

(22) Filed: May 28, 2025

Related U.S. Application Data
(60) Provisional application No. 63/652,857, filed on May 29, 2024.

(51) Int. Cl. A61B 17/72; A61B 17/04; A61B 17/86; A61F 2/00; A61F 2/08

(52) U.S. Cl. CPC A61B 17/725; A61B 17/0401; A61B 17/8625; A61B 17/863; A61B 17/8635; A61F 2/0811; A61B 2017/0438; A61B 2017/0464; A61F 2002/0081; A61F 2002/0086; A61F 2002/0817

(57) ABSTRACT

A three-dimensional (“3D”) printed orthopedic fixation implant can reduce stress shielding and/or enhance osseointegration. The implant can include: a head defining a proximal end of the implant; a tip defining a distal end of the implant; a core extending from the head to the tip, the core being elongate; and threads extending along at least a portion of the core. One or more functionally graded lattice structures can be propagated longitudinally and/or radially in at least portions of the core. A density of the one or more functionally graded lattice structures can have a varying longitudinal gradient and a varying radial gradient.

### INCORPORATION BY REFERENCE TO ANY PRIORITY APPLICATIONS

The present application claims priority benefit under 35 U.S.C. § 119 (c) to U.S. Provisional Patent Application Ser. No. 63/652,857, filed May 29, 2024, titled “Functional Gradient Lattice in Orthopedic Fixation Implant”, the disclosure of which is hereby incorporated in its entireties by reference herein. Any and all applications for which a foreign or domestic priority claim is identified in the Application Data Sheet as filed with the present application are hereby incorporated by reference under 37 CFR 1.57.

### BACKGROUND

### Field

The present disclosure relates generally to an orthopedic fixation implant with a porous structure to improve osscointegration and optimize mechanical properties. In particular, the present disclosure relates to a three-dimensionally (“3D”) printed bone screw, e.g., for use in fracture fixation, spinal fixation, or joint replacement implant stabilization, or anchor, e.g., for use in soft tissue repair to bone.

### Description of the Related Art

Orthopedic fixation implants have various applications. Bone screws are often used in orthopedic surgery to secure bone sections to each other (e.g., fracture fixation, spinal fusion) or to retain in place another implant (e.g., a plate, a prosthesis, a cap, etc.). The bone screws may have a screw head that can receive a driver tool and a threaded screw shaft for engaging the patient's bone. Suture anchors are often used for fixing tendons and ligaments to bone.

### SUMMARY

In some aspects, the techniques described herein relate to a three-dimensionally (“3D”) printed orthopedic fixation implant configured to reduce regional implant stress, mitigate stress shielding, and enhance osseointegration when inserted into bone of a patient, the implant including: a head defining a proximal end of the implant; a tip defining a distal end of the implant; a core extending from the head to the tip, the core being elongate; and threads extending along at least a portion of the core, wherein one or more functionally graded lattice structures can be propagated longitudinally and/or radially in at least portions of the core, a density of the one or more functionally graded lattice structures having a varying longitudinal gradient and a varying radial gradient.

In some aspects, the head can be a solid head without any lattice structure.

In some aspects, the one or more functionally graded lattice structures can be propagated further into the head.

In some aspects, the implant can be a suture anchor configured to secure a tendon or ligament to bone.

In some aspects, the tip can be a solid tip without any lattice structure.

In some aspects, the one or more functionally graded lattice structures can be propagated further into the tip.

In some aspects, the one or more functionally graded lattice structures can be propagated further into the threads.

In some aspects, the varying radial gradient density can be highest toward a center of the core and lowest toward a periphery of the core.

In some aspects, the varying longitudinal gradient density can be highest toward the head and lowest toward the tip.

In some aspects, the implant can be configured to be inserted unicortically with the head surrounded by cortical bone and the tip surrounded by cancellous bone.

In some aspects, the varying longitudinal gradient can be highest toward the head and the tip, and lowest at a location along the core between the head and the tip.

In some aspects, the implant can be configured to be inserted bicortically with the head and the tip surrounded by cortical bone and a mid-portion of the core surrounded by cancellous bone.

In some aspects, an outer shell of the core can be solid.

In some aspects, the one or more functionally graded lattice structures can extend through an outer surface of the core.

In some aspects, the core can include a solid inner core.

In some aspects, the implant can further include a longitudinal channel extending through the implant. In some aspects, the longitudinal channel may allow for passage of screw over a wire. In some aspects, the longitudinal channel may allow for instilling bone cement, cement substitute (e.g. calcium phosphate, magnesium-phosphate, etc.), and/or biologic factors to improve fixation and/or enhance osseointegration of the implant. In some aspects, the longitudinal channel may be closed off to a core (e.g., a lattice core). In some aspects, the longitudinal channel may remain open.

In some aspects, the one or more functionally graded lattice structures can include triply periodic minimal surface (TPMS) lattices.

In some aspects, the TPMS lattices can include one or more of gyroid, diamond, lidinoid, or splitP geometries.

In some aspects, the one or more functionally graded lattice structures can include a combination of different lattice geometries.

In some aspects, the one or more functionally graded lattice structures can include two or more functionally graded lattice structures that are interpenetrating or overlapping.

In some aspects, the one or more functionally graded lattice structures can include auxetic lattice structures.

In some aspects, the techniques described herein relate to a proximal femur fraction fixation kit including: the implant as disclosed above serving as a cephalomedullary nail lag screw; and an intramedullary implant.

In some aspects, the techniques described herein relate to a femoral neck fracture fixation kit, including: a plurality of the implants as disclosed above, wherein each of the plurality of implants can be cannulated.

In some aspects, the techniques described herein relate to a proximal humerus fraction fixation kit including: a plurality of the implants as disclosed above, the implant serving as a locking screw or a cortex screw; and a locking plate including a plurality of holes or apertures.

In some aspects, the techniques described herein relate to a spinal fixation kit including: a plurality of the implants disclosed above each serving as a pedicle screw or sacropelvic fixation screw; and a rod, each pedicle screw or sacropelvic fixation screw configured with a rod coupling head fastened to the rod to adjoin adjacent spinal segments.

In some aspects, the techniques described herein relate to a reverse shoulder arthroplasty kit including: the implant; and a reverse shoulder baseplate, the implant as disclosed above, the implant configured to engage a central hole of the baseplate.

In some aspects, the techniques described herein relate to a reverse shoulder arthroplasty kit, further including another one or more implants as disclosed above, the another one or more implants configured to be inserted into bone via one or more peripheral holes of the baseplate.

In some aspects, the techniques described herein relate to a rotator cuff repair kit including: a plurality of the implants as disclosed above.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1A illustrates a side view of an example orthopedic fixation implant of the present disclosure.

FIG. 1B illustrates a longitudinal cross-sectional view of an example orthopedic fixation implant of the present disclosure.

FIGS. 2A-2D illustrate various example unit cells of lattices in the orthopedic fixation implants disclosed herein.

FIGS. 3A-3E illustrate an orthopedic fixation implant with example variations in the inner core.

FIGS. 4A-4E illustrate an orthopedic fixation implant with example variations in the lattice component propagation.

FIGS. 5 and 5A-5E illustrate various views of an example screw with rectangular linear gradient gyroid lattice and a discrete custom-profile inner core.

FIGS. 6 and 6A-6E illustrate various views of an example screw with cylindrical linear gradient gyroid lattice and a natural gradient inner core.

FIGS. 7 and 7A-7E illustrate various views of an example screw with cylindrical quadratic gradient diamond lattice and an hourglass gradient inner core.

FIGS. 8 and 8A-8E illustrate various views of an example screw with rectangular gradient diamond lattice and a central throughbore or channel.

FIG. 9A illustrates a side view of an example cephalomedullary nail lag screw of the present disclosure.

FIG. 9B illustrates a detailed perspective view of the distal portion of the lag screw of FIG. 9A.

FIG. 10A illustrates a side view of an example cephalomedullary nail lag screw of the present disclosure.

FIG. 10B illustrates a detailed perspective view of the distal portion of the lag screw of FIG. 10A.

FIG. 11 illustrates an example application of a cephalomedullary nail lag screw of the present disclosure coupled with a cephalomedullary nail for use in proximal femoral fracture fixation.

FIGS. 12 and 12A-12D illustrate various views of an example cephalomedullary nail lag screw with a bidirectional gradient lattice inner structure.

FIGS. 13 and 13A-13E illustrate various views of an example cephalomedullary nail lag screw with a hybrid functional gradient lattice structure.

FIGS. 14 and 14A-14E illustrate various views of an example cephalomedullary nail lag screw with a hybrid functional gradient lattice structure.

FIG. 15 illustrates an example application of a screw of the present disclosure in reverse shoulder arthroplasty baseplate fixation.

FIGS. 16 and 16A-16E illustrate various views of an example reverse shoulder arthroplasty baseplate central screw with a functionally graded cylindrical gyroid lattice structure.

FIGS. 17 and 17A-17E illustrate various views of an example reverse shoulder arthroplasty central screw with a functionally graded cylindrical graphed diamond lattice structure.

FIGS. 18 and 18A-18E illustrate various views of an example reverse shoulder arthroplasty central screw with a functionally graded cylindrical splitp lattice structure.

FIG. 19 illustrates a side view of an example functionally graded gyroid lattice bone anchor.

FIGS. 20A-20D illustrate various views of an example functionally graded diamond lattice bone anchor.

FIGS. 21A-21C illustrate various views of an example bone anchor with superimposed hybrid lattice combination of diamond and splitP.

FIGS. 22A-22I illustrate steps of creating gradient latticing in an orthopedic fixation implant disclosed herein.

FIG. 23A illustrates a simulated progress of bone ingrowth into an orthopedic fixation implant disclosed herein.

FIG. 23B illustrates a simulated 100% bone ingrowth into an orthopedic fixation implant disclosed herein.

FIG. 23C illustrates simulated pull-out strength of an orthopedic fixation implant disclosed herein at 100% bone ingrowth.

FIGS. 24A-24C and 25A-25C illustrate finite element analysis (FEA) simulations of the impact of torsional load during insertion of a solid titanium screw (A), a non-graded lattice core screw (B), and a graded lattice core screw (C) at different insertion depths.

FIGS. 26A-26C illustrate FEA simulations of the impact of bending load during insertion of a solid titanium screw (A), a non-graded lattice core screw (B), and a graded lattice core screw (C) at a certain insertion depth.

FIGS. 27A and 27B illustrate FEA simulations of the impact of axial loading at the tip of a fully inserted solid titanium screw (A) and a graded lattice core screw (B).

### DETAILED DESCRIPTION

These and other features, aspects, and advantages of the present disclosure are described with reference to the drawings of certain embodiments, which are intended to schematically illustrate certain embodiments and not to limit the disclosure.

In surgical treatment for fracture stabilization (osteosynthesis), a bone screw is used to stabilize the fracture and/or supplementary implants (e.g. plate, intramedullary nail, etc.) in order to allow fracture union. In spinal fusion surgery, bone screws are used to anchor adjacent vertebral segments via interconnecting rods. In joint reconstructive procedures (e.g. hip arthroplasty, shoulder arthroplasty, etc.), bone screws are used to provide initial fixation of the implant to allow osseointegration and durable cementless fixation of the prosthetic components. With a screw (such as screws with a screw head, a variable thread pitch, and/or otherwise), one can compress the implant (e.g., metal plate) into the bone, thus providing compression for improved initial stability. However, traditional bone screws have their limitations. Traditional screws may lack porosity for encouraging bone ingrowth, and therefore may be complicated by micromotion and loosening over time that can precipitate screw migration, screw breakage, fracture non-union, and/or implant loosening and/or failure. Furthermore, traditional bone screws do not optimize mechanical strength properties to match regional bone density at insertion location, and thus create a stress riser as well as a stress-shielding phenomenon within the surrounding bone that can further precipitate peri-implant fracture, bone resorption, screw loosening, intra-articular screw penetration, and failure. These shortcomings of traditional bone screws are responsible, in part, for many of the most common hardware related complications seen in fracture fixation, spinal fusion, and total joint replacement, resulting in substantial patient morbidity and societal burden. For example, the most common complication of hip fracture fixation, as well as proximal humerus fracture fixation, is screw cut-out, resulting in intra-articular screw penetration and construct failure, resulting in progressive joint surface destruction and commonly requiring conversion to joint replacement as a salvage procedure. Additionally, in spinal fixation, screw pull-out and subsequent hardware failure is among the most common complications, while in reverse shoulder arthroplasty, implant loosening and stress fracture propagation at the screw tip is common. These hardware-related complications result in substantial morbidity related to pain, disability and revision surgery. Therefore, osseointegration can be desirable for many orthopedic devices and implants. The screws disclosed herein improves the bone screw by using a functional gradient lattice structure to enhance osseointegration, improve durable implant stability, optimize mechanical performance, diminish stress riser formation, and mitigate stress shielding within the surrounding bone.

Bone screw fixation can be achieved by primary fixation and secondary fixation. Primary fixation can be via the threads, and optionally the use of bone cement augmentation to interface between a portion of the bone screw and the bone surface, at the time of implant placement. Secondary fixation can be due to osseointegration, when a roughness of the implant surface and/or porosity of the implant allows for bone ongrowth or ingrowth to provide mechanical interlocking after implant placement. Osseointegration can be an important feature for many orthopedic devices and implants in improving implant stability.

Therefore, it can be desirable for a bone screw to include a certain amount of porosity to encourage osseointegration. The orthopedic fixation implants disclosed herein (including but not limited to screws and suture anchors) can be manufactured using 3D printing or other suitable techniques to include lattice structures in certain portions of the screw to promote osscointegration. The screws disclosed herein can optionally include solid threads for advancement through bone. In some embodiments, the orthopedic fixation implants disclosed herein can enhance osseointegration, minimize stress shielding, optimize mechanical performance, and/or mitigate implant and peri-implant fracture. In some embodiments, the orthopedic fixation implants disclosed herein can be additively manufactured, bidirectional functionally graded lattice screws or suture anchors with variable core density.

Further, many existing bone screws can be limited by stress-shielding and loosening, breakage, and/or stress-riser resulting in peri-implant fracture of bone. This limitation is in part due to mismatch in mechanical properties of the implant material, which may be titanium, stainless steel, or other types of suitable metal materials, to those of cortical and cancellous bones in which the screw is implanted. This relevance of this mismatch is emphasized by Wolff's law, which explains how bone resorption can occur around an excessively stiff screw, which leads to loosening and failure of the screw and associated implant (e.g. plate, nail, etc.) over time. For example, cortical bone may have a Young's modulus value of about 5 GPa to about 23 GPa and cancellous bones may have an even smaller Young's modulus value than cortical bone, e.g., about 0.01 GPa to about 1.57 GPa. In contrast, a commonly used titanium alloy for medical implants, Ti-6Al-4V, can have a Young's modulus value of about 114 GPa. Minimizing the difference between the Young's modulus values of the fixation implant and the bones can improve osseointegration and/or mitigate a stress-riser and the stress shielding phenomenon, resulting in improved durability of the fixation implant.

In some embodiments, additive manufacturing can generate an orthopedic fixation implant with an integrated lattice structure (e.g., additively manufactured triply periodic minimal surface (TPMS)). The orthopedic fixation implant manufactured using the additive manufacturing techniques disclosed herein can achieve mechanical properties that are more similar to the biologic structure of bone, decreasing the amount of material to optimize pore size and structure for osseous ingrowth, while retaining mechanical integrity and strength of the structure and improving energy absorption. Structures that can be produced using additive manufacturing can include lattice, TPMS (gyroid, diamond, primitive, lidinoid, splitP, and the like), hybrid/interpenetrating lattices, and the like. Lattice systems can broadly include cubic, triclinic, monoclinic, hexagonal, rhombohedral, orthorhombic, tetragonal, cubic; with lattice centering types that can include primitive, base-centered, body-centered, or face-centered.

The orthopedic fixation implant disclosed herein may be made of one or more biocompatible materials. In some embodiments, the biocompatible materials may include a resorbable material. Examples of such resorbable materials used for making an orthopedic fixation implant disclosed herein may include but are not limited: Magnesium Alloys, Polylactic acid (PLA), Polycarpolactone (PCL), Poly (lactic-co-glycolic acid) (PLGA), Tricalcium Phosphate (TCP), Hydroxyapatite (HA), or any combinations thereof. The resorbable material may be used for the entire orthopedic fixation implant or for certain portions of the implant (e.g., the threads, the outer portion of a core, an entire core, etc.). The definition of various portions of the implant are described in greater detail elsewhere in the present disclosure. In some examples, the outer portion of the core or the screw tip may be resorbable (such as by including a magnesium alloy) to further enhance osseointegration and improve load transfer (decrease stress-riser) at the tip of the implant (e.g., any of the screw examples disclosed herein). In some embodiments, a functional gradient (disclosed elsewhere herein) may be further modulated by the use of multiple materials in the implant.

### Overview of a Functionally Graded Lattice Orthopedic Fixation Implant

In the present disclosure, using additive manufacturing or otherwise, the orthopedic fixation implants disclosed herein can include functionally graded lattice to aid in matching regional implant stress and bone density patterns that are seen in vivo in order to mitigate stress-shielding, improve osscointegration, and/or improve durable fixation by preventing loosening and peri-implant bone resorption. In other words, functional graded lattice can have more desirable biomechanical properties compared to a uniform lattice. The gradient lattice architecture is tailored to optimize osseointegration near the screw-bone interface and enhance mechanical strength near high-stress regions (e.g., head-shaft junction). Additionally, gradient lattice can have improved energy absorption and fatigue resistance compared to a non-graded homogenous lattice material.

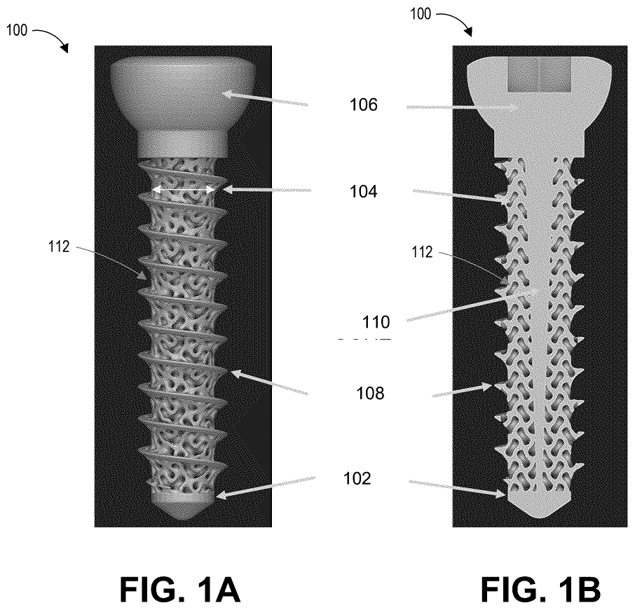

A bone screw can typically include different components as illustrated in FIGS. 1A and 1B. As shown, the screw 100 can include a tip 102 defining a distal end of the screw 100, a core 104 extending from the distal end toward a proximal end of the screw 100, and optionally a head 106 at the proximal end of the screw 100. The core 104 can be elongated. In the case of a headless screw, the proximal end of the core 104 defines the proximal end of the screw. Helical threads 108 can extend along an outer surface of the core 104. In the illustrated example, the core can optionally include a solid inner core 110 surrounded by lattice structures 112 in a remainder of the core 104. In other implementations, the inner core 110 may be hollow or may include lattice structures 112 at least along a portion of the inner core 110.

Although the terminology of different portion of the orthopedic fixation implant is described with reference to a screw, the same terminology can be applied to other types of orthopedic fixation implants disclosed herein. Head is defined as the portion of the fixation implant that allows for seating of a driver to advance the implant forward, and, when present, is defined as the proximal end of the implant. The head may have a larger diameter than the core (for example, to allow for compression upon implant insertion), or may have the same diameter as the core (for example, to allow for countersinking of the implant). Head refers to the side of the implant opposite the tip of the implant along a longitudinal axis regardless of head geometry. Tip is defined as the side of the implant that first enters the bone, and is defined as the distal end of the implant.

The orthopedic fixation implant described herein can be 3D printed or additively manufactured. The 3D printed core (or other parts of the implant) can include but are not limited to lattice, TPMS (gyroid, diamond, primitive, lidinoid, splitP, and the like), hybrid/interpenetrating lattices, and the like. The 3D printed structure can have gradients. The functionally gradient lattice may include: (a) longitudinal gradient, e.g., decreasing density from the head to the tip; and/or (b) radial gradient, e.g., increased density toward or at the center of the core, i.e. an inner core, with decreased density/larger pores toward or at periphery/surface of the core. Such implants may be used, for example, in fixation of proximal femur fractures or in stabilization of reverse shoulder arthroplasty baseplates, or in other orthopedic applications as disclosed herein. The functional grading of the lattice structures in the fixation implants disclosed herein may include unidirectionally graded lattice or preferably bidirectionally graded lattice, or other multi-directionally graded lattice. Although the descriptions regarding the different types of lattice structures below are with reference to screws, the features of the lattice structures disclosed herein are also applicable to other types of orthopedic fixation implants, such as suture anchors.

A unidirectionally graded lattice can be a radially graded lattice or a longitudinally graded lattice. For example, a screw with a radially graded lattice can have decreasing density from the center of the core outward toward a periphery of the core in a radial direction. Such a radially graded lattice can enhance osseointegration circumferentially by optimizing pore size for osseointegration and more similarly replicating the density of surrounding bone, and/or lead to improved mechanical properties by nature of improved energy absorption and strength in gradient lattice structures. Greater density towards the center of the core can maintain overall strength of the screw to enable insertion under high torque and prevent implant breakage under loading. A longitudinally graded lattice can have varying density along a length of the core, which can better match the mechanical properties of the surrounding or regional bone in which the fixation implant is inserted. A reduced density near the tip can a) prevent a stress riser at the tip, which can prevent peri-implant fractures, and/or b) prevent cut-out of fixation implant through the bone, particularly in osteopenic bone. Additionally, achieving ingrowth of the fixation implant over time can minimize risk of implant failure. The porosity of the implant can also allow for greater potential surface area for fracture healing via pores that interconnect the two segments of apposed bone. Traditional bone screws provide fixation across a fracture site to allow healing, although also occupy the fracture site with a biologically inert object thus diminishing the effective surface area a fracture has to heal. A functionally graded lattice screw optimized for osscointegration may therefore improve fracture healing by enhancing angiogenesis and nutrient exchange, and therefore mitigate risk of fracture nonunion and avascular necrosis. The interconnection of apposed bone segments improves the overall retention strength of the bone as well as the position and orientation fixation of the screw.

In some implementations, a longitudinally graded lattice may be more tailored for a cancellous screw application. The lattice for the cancellous screw application can have increased density (thicker walls and/or smaller pores) towards the head, which is nearest to the cortical bone, and decreased density (thinner walls and/or larger pores) towards the tip, which is nearest to cancellous bone, along a longitudinal axis. The lattice for the cancellous screw application can thus achieve more similar modulus of elasticity to surrounding bone, which may include both cancellous and cortical bones. That way, the lattice for the cancellous screw application can enhance osscointegration, prevent stress riser at tip (i.e. peri-implant fracture or implant cut-out), and/or prevent stress shielding of bone, while accounting for in vivo physiologic strain (that is, with higher strain towards the head).

In some implementations, a longitudinally graded lattice may be more tailored for a cortical screw application. The lattice for the cortical screw application can have increased density (thicker walls and/or smaller pores) towards head and tip, which are both nearest to the cortical bone, and decreased density (thinner walls and/or larger pores) towards a mid-portion of the core, which is nearest to the cancellous bone, along longitudinal axis. The lattice for the cortical screw application can thus achieve more similar modulus of elasticity to surrounding bone, which can enhance osseointegration, prevent stress riser at the tip (i.e. peri-implant fracture or implant cut-out), and/or prevent stress shielding of bone, while accounting for in vivo physiologic strain (that is, higher strain towards the head and the tip).

A bidirectional lattice can have varying density in more than one direction, for example, in both the radial and longitudinal directions, a transverse direction, and/or an oblique direction, or otherwise. A bidirectional lattice can result in significantly higher compressive modulus, yield stress, and/or plateau stress compared to a unidirectional gradient. This can be due to the bidirectional lattice combining the advantages of both the radially graded lattice and the longitudinally graded lattice. As a non-limiting example, a functionally gradient lattice may include: (a) decreased density at the tip; and/or (b) increased density toward or at center of the core with decreased density toward or at the periphery/surface of the core. As described above, a functionally graded fixation implant with reduced density near the tip can more similarly replicate the density of local or regional bone. The density variation in the radial direction can improve the strength of the fixation implant.

The variation in density can be due to at least varying wall thickness and/or varying pore sizes as well as the type of lattice structure and the density of the cell map throughout which each voxel is patterned. In non-limiting examples, a gradient in an orthopedic fixation implant can be achieved by one or more of: a) varying porosity, wall thickness, and/or cell size; b) cell distribution map; c) material composition changes; d) tapered core (longitudinal gradient); and/or e) solid core (radial gradient). The gradient is achieved in at least one or preferably at least two of the cylindrical coordinate planes (longitudinal and radial planes).

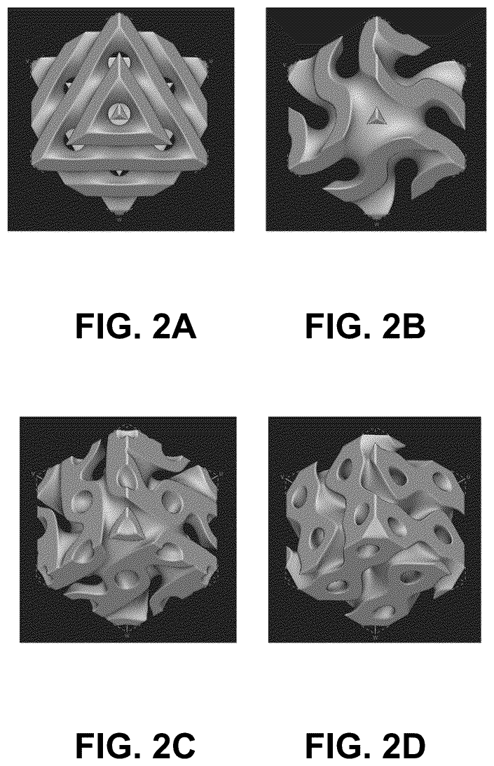

In non-limiting examples, the lattice geometry can encompass a combination of different lattice types and/or centering. Lattice types can include but are not limited to tetragonal, monoclinic, orthorhombic, hexagonal, triclinic, trigonal, or cubic. Lattice centering can include but are not limited to primitive, base-centered, body-centered, or face-centered. In some implementations, the lattice structures in the present disclosure can include triply periodic minimal surface (TPMS) lattices. Non-limiting examples of TPMS lattices can include but are not limited to gyroid, diamond, Schwarz primitive, lidinoid, splitP, or the like. FIGS. 2A-2D illustrate some non-limiting example unit cells of TPMS structures. FIG. 2A illustrates a Diamond lattice. FIG. 2B illustrates a Gyroid lattice. FIG. 2C illustrates a Lidinoid lattice. FIG. 2D illustrates a SplitP lattice. In some implementations, the lattices can include a hybrid lattice, which can combine different lattice geometries, or include interpenetrating, superimposed, and/or overlapping lattices.

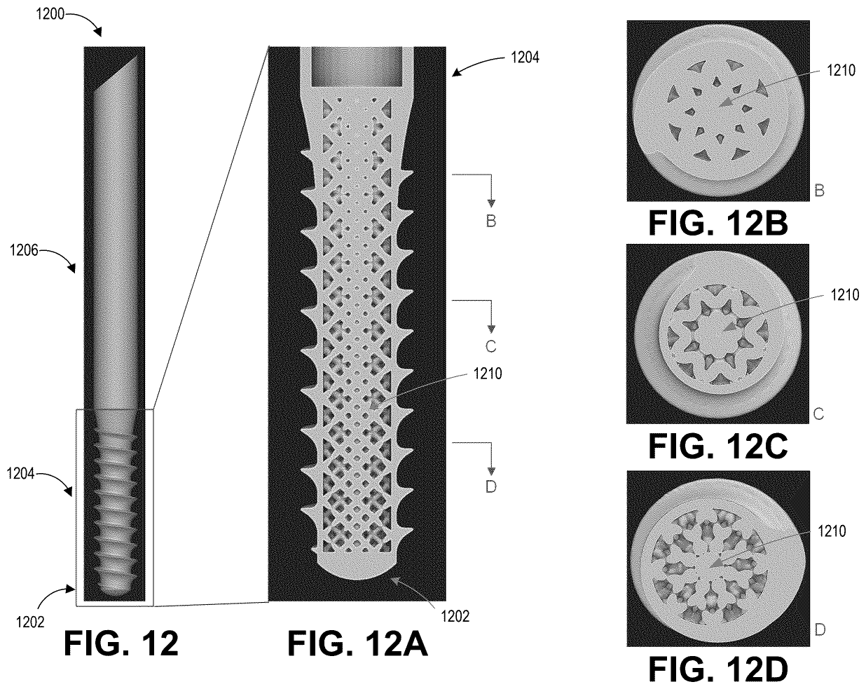

In some implementations, the orthopedic fixation implant disclosed herein can include solid shells (see, e.g., FIG. 12A) in at least a portion of the implant. In non-limiting examples, a solid shell may be distributed along the tip to aid in advancement, or can be applied to the entire exterior surface of the core, head, or portion thereof, of the fixation implant. The solid shells may prevent host bone contact with the lattice structure in situations where bone ingrowth is undesirable in all or certain portions of the fixation implant. Similarly, in some implementations, the lattice geometry may include entire fixation implant (including the head, the core, the threads, and the tip), or just a portion or portions thereof.

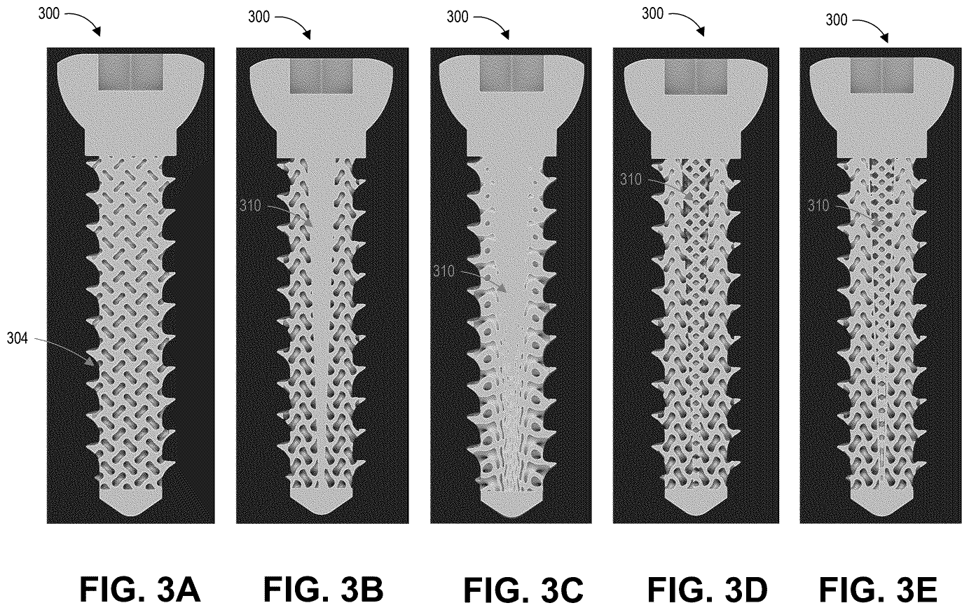

FIGS. 3A-3E illustrate longitudinal cross-sectional views of different orthopedic fixation implants to demonstrate the modifiability of the inner core 310 of the fixation implant 300. The implant 300 in FIG. 3A includes a core 304 with a unidirectionally graded lattice (with the density decreasing from the head toward the tip) for illustration purposes. The inner core features shown in FIGS. 3A-3E can be incorporated in fixation implants with any other types of lattice structures, including but not limited to any bidirectional lattice structures. In FIG. 3A, the unidirectionally graded lattice extends throughout the entire core 304 such that there is no distinct inner-core feature. The distal portion of the core 304 and the tip can be surrounded by cancellous bone and the head can be embedded in cortical bone. In other words, the gradation of the density reflects the density of the surrounding bone such that the screw has optimization for osscointegration as well as minimizing stress shielding.

In order to achieve a multi-directional gradient of the implant, which can enhance structural integrity and energy absorption of lattice structures, the inner core of the implant can be either independently incorporated as a custom core profile as disclosed in FIG. 3B or can be integrated within the global geometry of the lattice as represented in the cylindrical lattice structures disclosed in FIG. 3C. The inner core 310 can allow for enhanced modularity of the implant design in terms of multi-material design as well as hybrid lattice design. Such designs can enhance the strength of the orthopedic fixation implant 300 without compromising the optimization for osscointegration at the periphery of the implant 300. In embodiments such as shown in FIGS. 3B, 3D, 3E, the inner core 310 can include a custom profile. In a non-limiting example such as shown in FIG. 3B, the custom profiled inner core 310 can be solid. In one embodiment such as shown in FIG. 3D, the custom profiled inner core 310 can include an open lattice. The open lattice design can enable bone growth all the way through the inner core 310, and can reconnect to bone growing through the implant from the other side in the radial direction. In one embodiment such as shown in FIG. 3E, the custom profiled inner core 310 can include a walled lattice. The inner core 310 in FIG. 3E adds a solid wall between the inner core 310 and the remainder of the core 304. The solid wall can biomechanically strengthen the inner core 310, acting as a strut within the lattice structure of the core 304 and providing a solid surface for the lattice structures in the core 304 to terminate at. In some embodiments, the solid wall can also accommodate a hollow inner core that results in a cannulated screw such that a guidewire can be used for alignment. In some aspects, the hollow inner core of the cannulated screw may perform the functions of a longitudinal or central channel disclosed elsewhere herein.

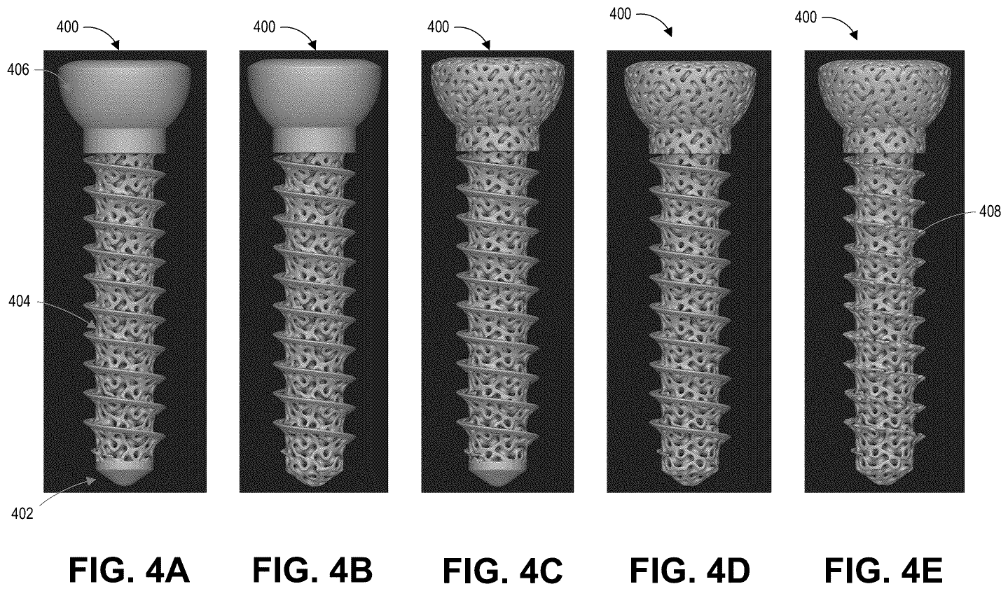

As described elsewhere in the present disclosure, the lattice structure can extend to any one or more portions of the orthopedic fixation implant, including but not limited to the tip, head, core, inner core, and/or the threads. FIGS. 4A-4E illustrate side views of non-limiting examples of various functionally graded orthopedic fixation implants to demonstrate the modifiability and/or flexibility in terms of distribution of the lattice structure in one or more parts of the implant. For illustration, the implant 400 is shown with the highest density at the head 406 and the least density towards the tip 402 and the periphery of the implant 400. However, the modifiability of the lattice structure is applicable to any orthopedic fixation implant with any types of functionally graded lattice structures disclosed herein. As shown in FIG. 4A, the lattice structure can extend to or be propagated through only the core 404, with the head 406, the tip 402, and the threads 408 being solid. As shown in FIG. 4B, the lattice structure can extend to or be propagated through only the core 404 and the tip 402, with the head 406, and the threads 408 being solid. As shown in FIG. 4C, the lattice structure can extend to or be propagated through the core 404 and the head 406, with the tip 402 and the threads 408 being solid. As shown in FIG. 4D, the lattice structure can extend to or be propagated through the core 404, the head 406, and the tip 402, with the threads 408 being solid. As shown in FIG. 4E, the lattice structure can extend to or be propagated through the entire implant 400, including the core 404, the head 406, the tip 402, and the threads 408.

Lattice may be propagated through various components of the implant, and each of the different components of the implant may be fabricated with a different lattice structure or different lattice density in order to achieve specific functions. With increasing propagation of the lattice structure through various components of the orthopedic fixation implant, there can be increasing surface area for osscointegration and thus improve strength of biologic fixation of the implant to the bone. One benefit of incorporating lattice on the head is that this portion of the implant is typically embedded in dense cortical bone and thus can be an excellent location for the implant to integrate within the densest bone along the insertion path of the implant. For example, in the case of percutaneous pinning of a femoral neck fracture, a common complication is backing out of the screw and shortening of the fracture. Incorporating osseointegration into the head can mitigate backout and failure of the implants.

Additionally lattice on top of the head can be optimized for tendon ingrowth, such as in the implementation of bone anchors, which are commonly used for tendon-to-bone healing. In this implementation, a lattice head can provide another location for the tendon to heal. In some implementations, the head of the orthopedic fixation implant (e.g., a suture anchor) can additionally integrate any of the lattice structures disclosed herein (for example but not limited to TPMS lattice) for tendon ingrowth. In some implementations, such as in the implementation of a suture anchor, the implant can include an optional aperture within the implant to serve as a suture loading point.

In some implementations, the lattice structure in the orthopedic fixation implant can include an auxetic lattice. In other words, the lattice can have a negative Poisson's ratio and thickens when being stretched.

In some implementations, underneath a 3D printed core surface, the core may have a solid inner core or inner core with lattice structures to strengthen screw and prevent breakage. In some implementations as disclosed herein elsewhere, the orthopedic fixation implant may be cannulated. In other words, a central channel or throughbore may be present to allow for placement of screw over a wire or guidewire. In some aspects, the longitudinal or central channel may allow for passage of screw over a wire (e.g., a guidewire). In some aspects, additionally or alternatively, the longitudinal or central channel may allow for instilling bone cement, cement substitute (e.g. calcium phosphate, magnesium-phosphate, etc.), and/or biologic factors to improve fixation and/or enhance osseointegration of the implant. In some aspects, the longitudinal or central channel may be closed off to a core (e.g., a lattice core) in a certain region. In some aspects, the longitudinal or central channel may remain open to control cement/cement substitute/biologic factors and/or the like into specific regions. For example, a midportion (between the proximal and distal ends thereof) of screw to have connection to the lattice for biologic factors to be placed in region of bone healing or fusion. As another example, the tip portion of screw may have a central channel connected to the lattice core for cement/cement substitute to be placed to interdigitate with surrounding cancellous or osteopenic bone.

In some implementations, the orthopedic fixation implant can be tapered to allow easier insertion. The implant can include a tapered core with threads of a uniform outer diameter thread or both a tapered core and tapered thread (e.g., tapering towards the tip). In some implementations, the orthopedic fixation implant can include a self-tapping tip and/or can be self-drilling. In some implementations, the orthopedic fixation implant can include standard threads or reverse threads to be able to interlock the fixation implant into a plate implant. Optionally the fixation implant may be partially threaded to allow compression. The orthopedic fixation implant can be made of materials including but not limited to metals, metal alloys, polymers. The materials used in the orthopedic fixation implant can include biodegradable materials.

In some implementations, the external/major diameter of the orthopedic fixation implant can range from about 2 mm to about 17 mm. In some implementations, the core diameter can range from about 1 mm to about 13 mm. In some implementations, the length of the implant can range from about 5 mm to about 200 mm. In some embodiments, the thread pitch of the orthopedic fixation implant can be greater than about 1 mm, or higher.

In some implementations, the 3D printed structure can have a pore size of about 200 to about 750 μm. In some implementations, the 3D printed structure may have a strut thickness of about 200 μm to about 500 μm. In some implementations, the 3D printed lattice structure may have a porosity of about 50% to about 90%.

The orthopedic fixation implants disclosed herein can be used in various clinical applications. For example, a cephalomedullary nail for hip fracture treatment has a large head screw with most common complication being screw cut-out through osteopenic bone; a proximal humeral plate and screw fixation have common complications of bone resorption, intra-articular penetration of screws, and/or failure of fixation; currently, shoulder arthroplasty glenoid component has central fixation that is limited to screw or an ingrowth post; a suture anchor may experience pull-out and failure to enhance tendon to bone healing. The orthopedic fixation implant disclosed herein can address one or more of the shortcomings of the existing procedures described herein.

Examples of application of the screw disclosed herein can include but are not limited to: cephalomedullary nail lag screw (for example, with a screw of about 11 mm in diameter and a length of about 70 mm to about 130 mm); proximal humerus plate screws; pedicle screws; reverse shoulder arthroplasty baseplate screws, acetabular cup screws for total hip arthroplasty implant; suture or bone anchors; or any other functionally graded orthopedic fixation implants, for example, with higher density proximally and lower density distally or otherwise, to improve osscointegration, prevent stress shielding and mitigate peri-implant fracture.

In one example, the orthopedic fixation implant can serve as a screw that is coupled to a plate in a reserve shoulder application, such as reverse shoulder arthroplasty baseplate central screw and/or reverse shoulder arthroplasty baseplate peripheral screws. The reverse shoulder arthroplasty baseplate central screw disclosed herein can include a head optionally locking into plate while also allowing apposition of lattice on the screw and the backside of the baseplate to allow for bridging osseointegration between the host bone, the screw, and the baseplate. The orthopedic fixation implant can additionally or alternatively serve as baseplate peripheral screws (locking and/or non-locking) in reverse shoulder arthroplasty.

### Example Bone Screws

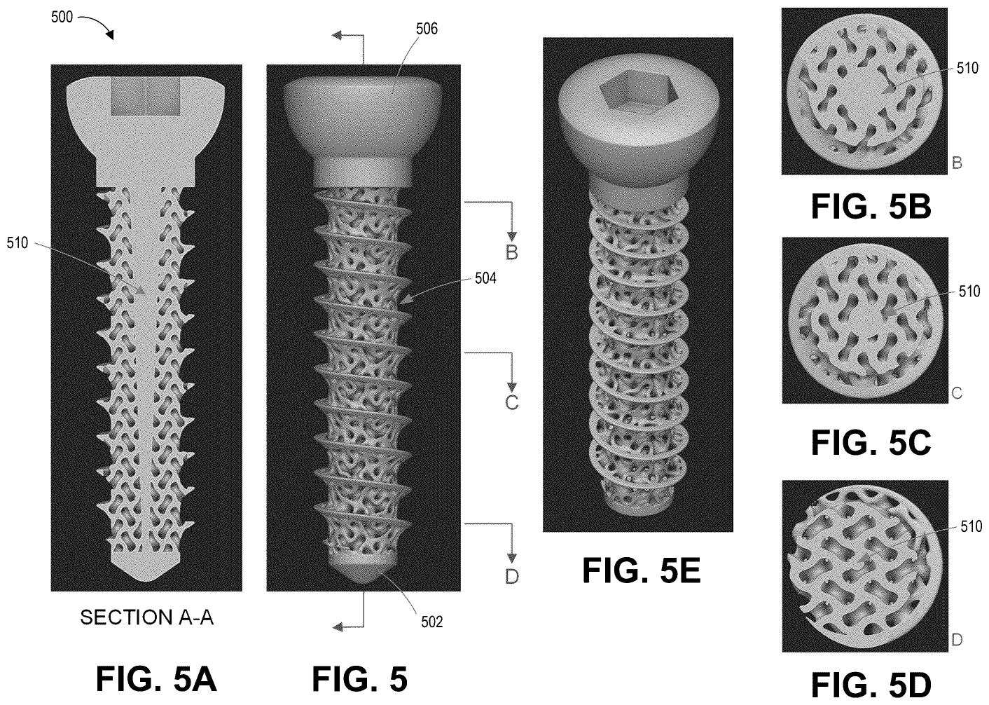

Various examples of bone screws will now be described with reference to FIGS. 5 and 5A-5E through FIGS. 8 and 8A-8E. These examples are not limiting and other combinations of lattice features are possible based on the disclosure herein. Features of the bones screws in these figures can be incorporated into one another. Features of the bone screws in these figures can also be incorporated into other examples of the orthopedic fixation implant disclosed herein. The bone screws in these figures can also incorporate features of other examples of the orthopedic fixation implant disclosed herein.

As shown in FIGS. 5 and 5A-5E, the bone screw 500 can include bi-directional functional grading (for example, linear) of rectangular gyroid structure. As shown, in the longitudinal direction, the bone screw 500 can have the highest density towards the head 506 of the screw 500 (see FIG. 5B) and the lowest density towards the tip 502 of screw 500 (see FIG. 5D). In the radial direction, the bone screw 500 can have the highest density towards a center of the core 504 of the screw 500 and the lowest density towards a periphery or outer surface of the screw 500. FIG. 5A illustrates a linear gradient in a longitudinal direction with the addition of a solid inner core 510 that tapers from the head 506 to the tip 502 of the screw 500 such that there is an additional gradient of density in the radial direction. The tapered inner core 510 is also referred to as a discrete custom-profile inner core. A custom profile refers to the ability to modify the inner core structure and size independent of the relationship to the outer core, so that the inner core can be optimized in terms of strength (or other mechanical properties) for the specific task/function the implant is being used for, independent from optimizing the outer core (which can typically be for purposes of osscointegration, stress shield mitigation, etc.). Having a dense inner core of the screw can improve the mechanical strength of the screw to prevent screw breakage, while a less dense periphery of the screw can achieve a more similar modulus of elasticity to the surrounding bone, which therefore can improve osseointegration and limit stress shielding.

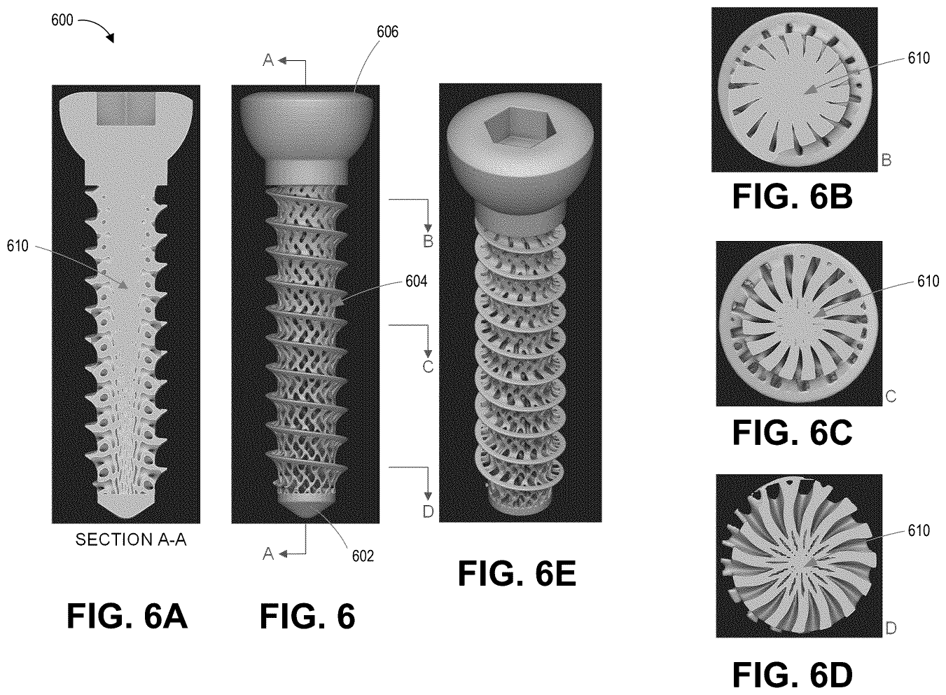

FIGS. 6 and 6A-6E illustrate a bone screw 600 with a cylindrical functionally graded (for example, linear) gyroid structures. The screw 600 can have the same bi-directional functional grading as the screw 500, that is, with the highest density at the head 606 and the center of the core 604 of the screw 600 and the least density towards the tip 602 and periphery of the screw 600. The solid inner core 610 includes a natural gradient inherent to the cell mapping and properties of cylindrical TPMS structures. Natural gradient core refers to the natural density gradient that exists with a cylindrical lattice pattern, such that the center of the core (inner core) has higher density than the periphery (outer core). When the lattice is functionally graded in the longitudinal axis/direction, there is a corresponding gradient in the radial direction that is propagated in the radial direction given the nature of a cylindrical gradient lattice.

The TPMS screw 500, 600 can have a cancellous screw application. The bi-directional functional gradient can advantageously have the highest density towards the head of the screw as the head of the screw is designed to be embedded within cortical bone, and the lowest density towards the tip of the screw as the core of the screw is designed to be embedded within cancellous bone. Additionally, the radial gradient with the most dense portion of the lattice along the central, longitudinal axis and the least dense portion being peripheral can advantageously have a more dense core or center for increased mechanical strength of the screw to prevent breakage, while the less dense portion of the external/peripheral part of the core can allow for osscointegration and better matching of the modulus of elasticity of the surrounding cancellous bone. Additionally there can be improvements in energy absorption and mechanical strength of bidirectionally graded lattices, which can further aid in preventing screw breakage and failure under loading conditions.

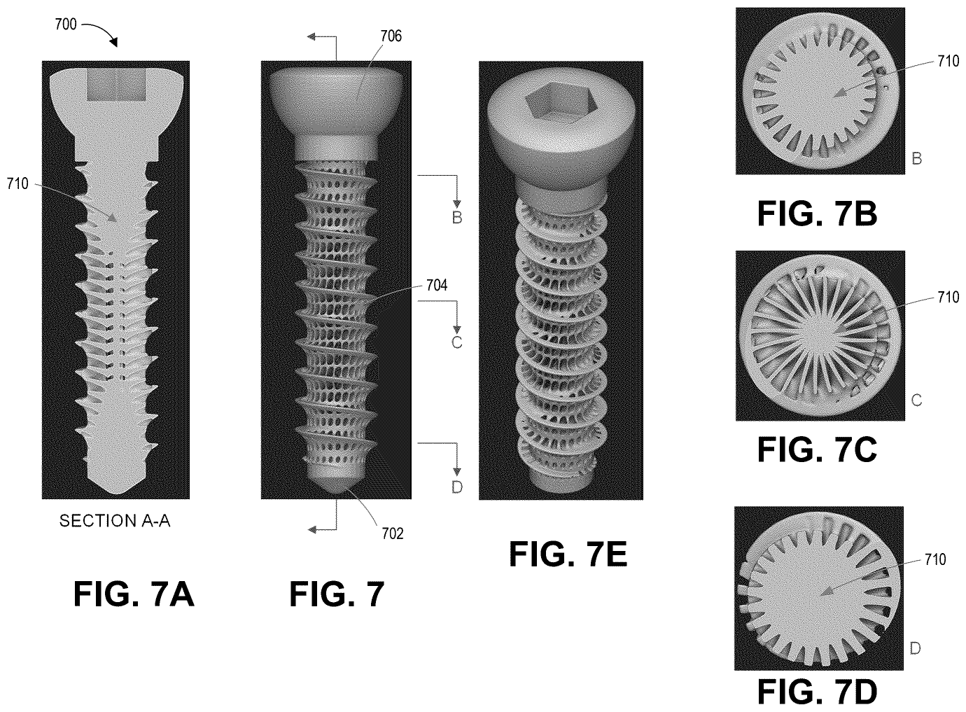

FIGS. 7 and 7A-7E illustrate a cylindrical diamond functionally graded (for example, quadratic) screw 700 with a highest density towards the head 706 (FIG. 7B) and the tip 702 (FIG. 7E) of the screw 700 and the least density towards the mid-portion (between the head 706 and the tip 702, see, e.g., FIG. 7C) of the screw 700 as well as the periphery of screw 700. As shown in FIG. 7A, the solid inner core 710 can include an hourglass gradient.

The screw 700 with the bi-directional functional gradient can have a cortical screw application. The lattice has the highest density towards the head and tip of the screw as the head and the tip of the screw are designed to be embedded within cortical bone (bicortical application), and the lowest density towards the mid portion longitudinally, which is designed to be embedded within cancellous bone. Additionally, there is a radial gradient with the most dense portion of the lattice being at the center of the core and the least dense portion being peripheral such that the more dense center of the core can allow for increased mechanical strength of the screw to prevent breakage, while the external/peripheral part of the core can allow for osscointegration and better matching of the modulus of elasticity of the surrounding cancellous bone. Additionally, there can be improvement in energy absorption and mechanical strength, as well as minimization of stress concentration of bidirectionally graded lattices, which can further aid in preventing screw breakage and failure under loading conditions. Moreover, the denser/solid tip can increase boring strength and robustness of the screw during insertion, which can be advantageous for being inserted into areas with the densest and most rigid bone, while still allowing for osscointegration.

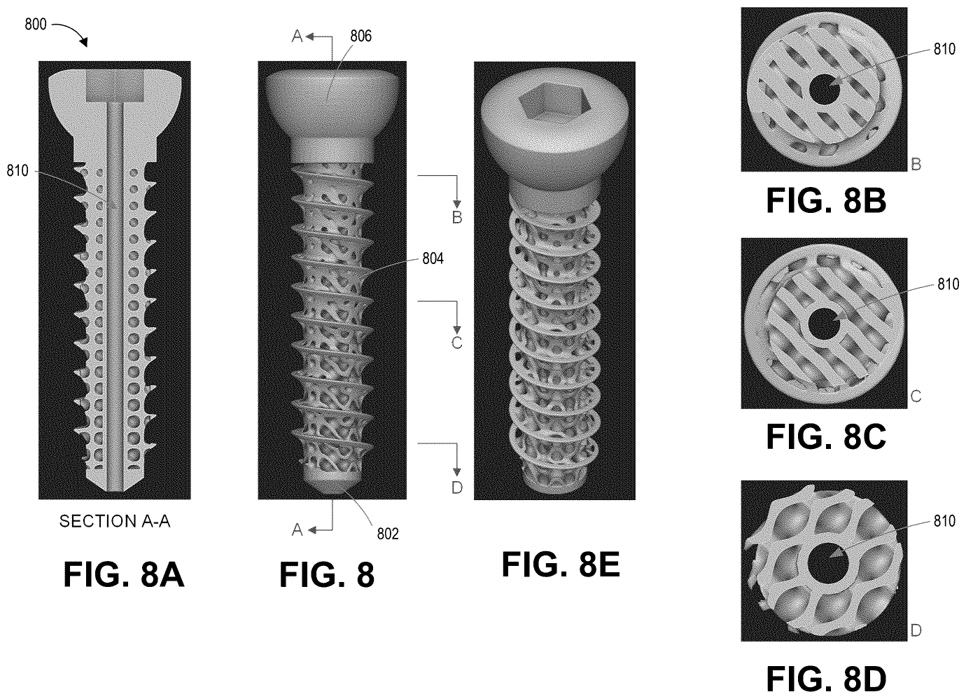

FIGS. 8 and 8A-8E illustrate a rectangular diamond functionally graded screw 800, with the highest density at the head 806 of the screw 800 and the least density towards the tip 802 of the screw 800. This screw 800 can include a hollow central throughbore or channel 810 through the entire length of the screw 800. The central bore 810, also referred to as cannulation, can allow for installation of medication or cement augmentation or other clinically relevant product through the central bore 810, and/or for installing the screw 800 via a guidewire.

### Example Fracture Fixation Application

In some implementations, the orthopedic fixation implant disclosed herein can be used in fracture fixation. As a non-limiting example, in intramedullary fixation of proximal femoral fracture, the orthopedic fixation implant disclosed herein can serve as a cephalomedullary nail lag screw.

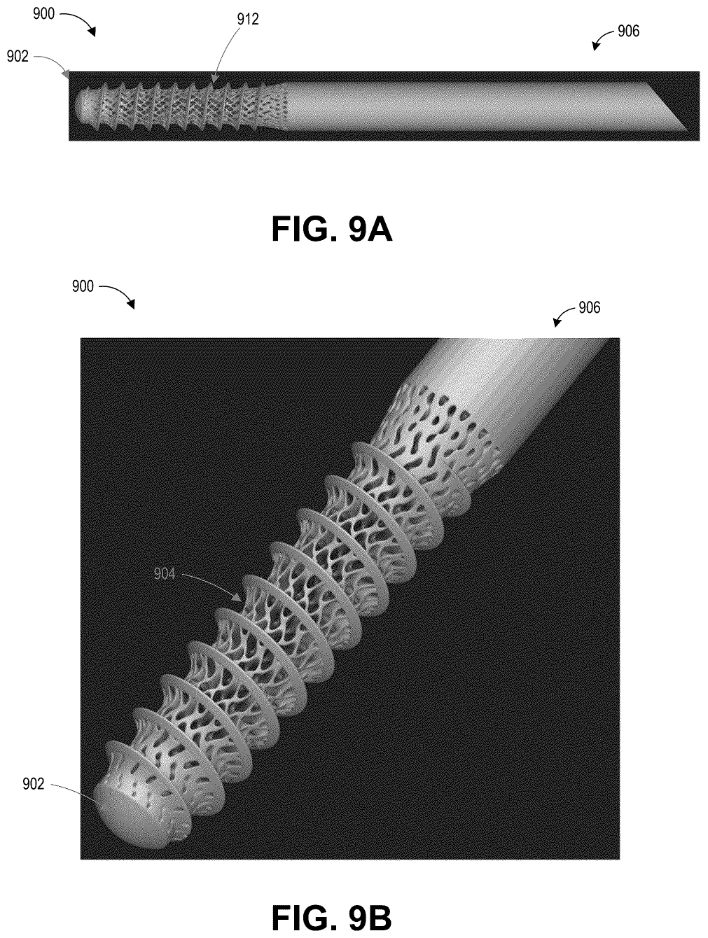

FIGS. 9A and 9B illustrate an exemplary cephalomedullary nail lag screw 900 including a head 906, a core 904, a tip 902, and threads 912 extending along the length of the core 904. The lag screw 900 can include an hourglass, bidirectional lattice structure at least along the length (for example, the entire length) of the core 904. In other words, the screw 900 can have the highest density at the head 906, the tip 902 and the center of the core 904, and the lowest density in the mid portion of the core 904 and the outer periphery of the core 904. The head 906, tip 902 and the threads can be solid. Inherent to a cylindrical lattice structure, the natural inner core may exemplify an hourglass profile, reflecting the gradient of the core 904; similar to the core 704 as shown in FIG. 7.

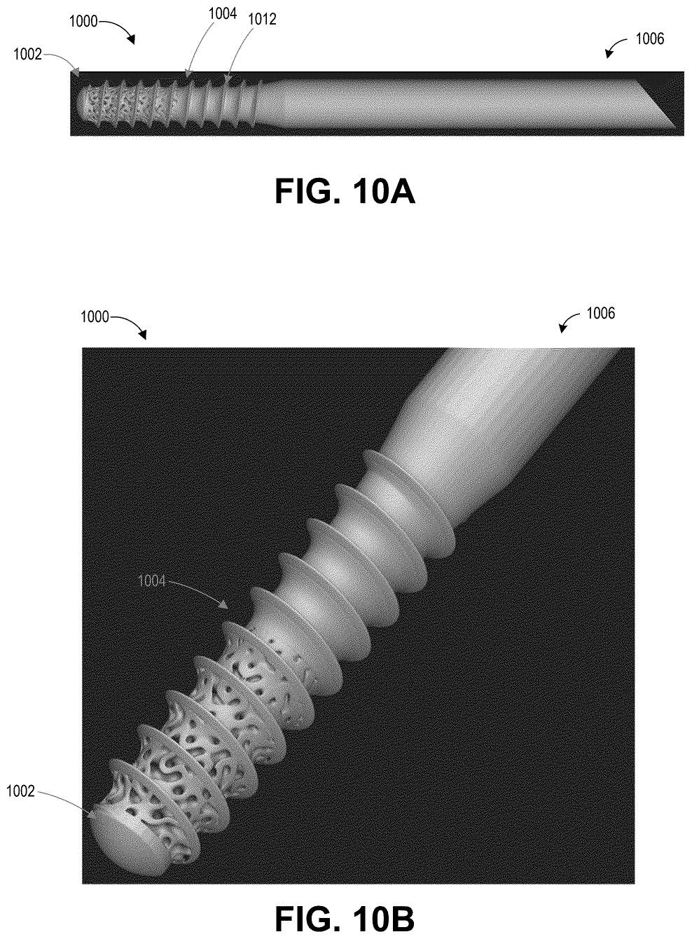

FIGS. 10A and 10B illustrate an exemplary cephalomedullary nail lag screw 1000 including a head 1006, a core 1004, a tip 1002, and threads 1012 extending along the length of the core 1004. The lag screw 1000 can include a functional gradient lattice structure along the length (for example, at least a partial length) of the score 1004. The functional gradient can include the lowest density of the screw towards the tip 1002 and the highest density towards the head 1006 (for example, at a location between the tip 1002 and the head 1006). The lattice structure only covers a portion (for example, a distal portion) of the core 1004 in FIGS. 10A and 10B, although in other embodiments, the lattice structure can cover the entire core and/or the entire screw.

In the treatment of proximal femoral fracture using intramedullary fixation, a common mode of failure for a traditional cephalomedullary nail lag screw is cut-out of the screw resulting in intra-articular penetration, which causes substantial morbidity and typically requires conversion to hip replacement as a next mode of treatment. This complication can be attributed to the substantial disparity between the modulus of elasticity of the screw and the surrounding bone, as well as the absence of osscointegrative capacity of traditional screws. Biomechanically, a screw with a graded decrease in density towards the periphery and the tip of the screw can mitigate stress-shielding and risk of implant cut-out and failure. A screw including a functionally graded lattice such that there are areas towards the tip of the screw that have decreased density (the screw 900, 1000) can more similarly match the surrounding native femoral head bone. Screw 900 depicts an hourglass gradation in density. Although the screw 900 is shown with increased density at the tip 902 of the screw 900, the screw 900 overall has a decreased density towards the distal end of the screw 900 compared to the proximal portion the core 904 of the screw. The hourglass gradient may advantageously aid in screw insertion as well as improving the stress dissipation between the screw tip 902 and the core 904 to prevent screw breakage. The length of the screw containing the lattice structure can be variable, for example, depending on the fracture location and/or a bone density pattern for a given patient. In a patient that is more osteoporotic, having a longer portion of the screw and/or for a greater surface area of the screw with a lattice structure in contact with the surrounding bone can be advantageous for improving osscointegration, fracture healing, and long-term implant stability. The lag screws disclosed herein can therefore be advantageous in improving treatment success of intramedullary fixation.

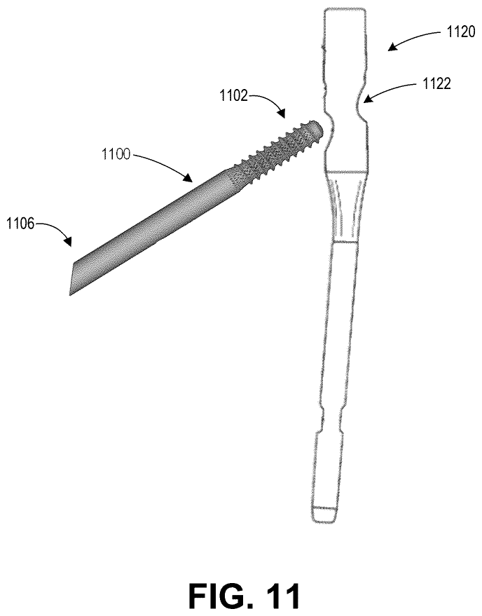

FIG. 11 illustrates the lag screw 1100 (which can be the screw 900 of FIGS. 9A-9B or any other lag screw examples disclosed herein) with an intramedullary implant (such as a nail) 1120. After the intramedullary implant 1120 is inserted into a proximal portion of the femur, the lag screw 1100 can be inserted into an aperture 1122 of the intramedullary implant 1120 from the tip 1102 of the lag screw 1100. Another screw (not shown) can be placed through the top of the intramedullary implant 1120 as oriented in FIG. 11 that interlocks into a groove of the lag screw 1100 to lock the intramedullary implant 1120 in place. The intramedullary implant 1120 and the screw 900 can be part of a fracture fixation kit.

FIGS. 12 and 12A-12D illustrate an exemplary cephalomedullary nail lag screw 1200. The screw 1200 can include a solid peripheral wall (that is, without lattice) at the exterior surface of the core 1204. An interior part of the core 1204 can include a bidirectional lattice structure. The solid external surface can prevent osseointegration while the inner lattice structure can maintain a functionally graded density of the screw. In the longitudinal direction, the interior of the core 1104 can have the highest density towards the head 1206 of the screw 1200 (see FIG. 12B) and the lowest density towards the tip 1202 of screw 1200 (see FIG. 12D). In the radial direction, the screw 1200 has the highest density towards a center of the core 1204 and the lowest density towards the solid peripheral wall of the core 1204.

The functionally graded lattice in the screw 1200 can reduce the amount of material use for the screw and/or the graded density can match the surrounding bone in order to prevent stress shielding of the surrounding bone and mitigate cut-out of the screw through the femoral head bone. The solid shell at the periphery of the core, by preventing osseointegration, may be beneficial for treating a younger patient. This is because the solid shell can simplify implant removal and make the removal process less morbid, which is important for the younger patient where revision internal fixation and osteosynthesis is favored over prosthetic joint replacement in the setting of complication (e.g., nonunion of the fractured bone).

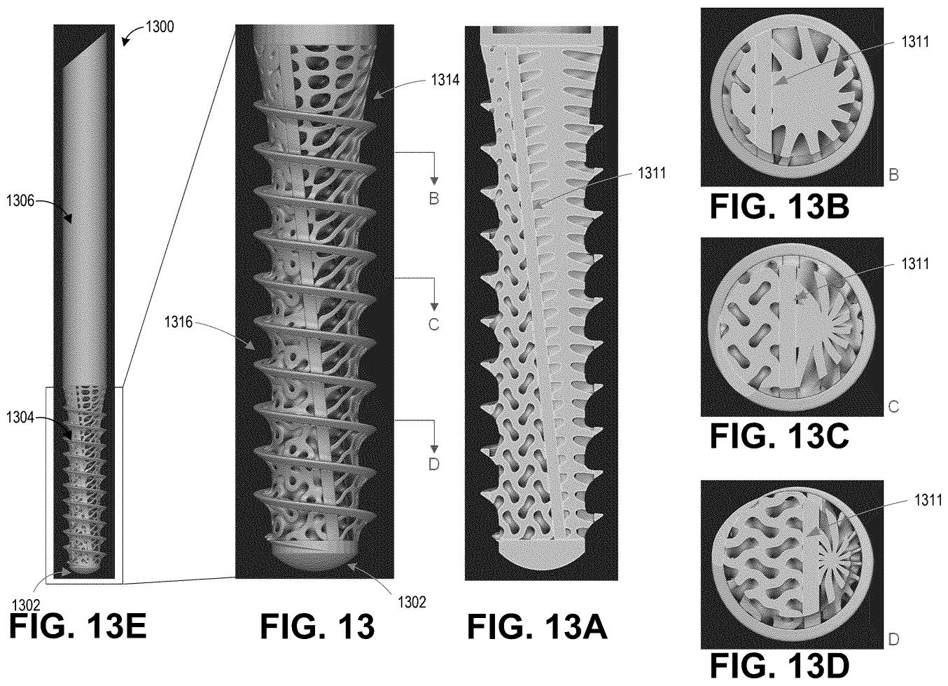

FIGS. 13 and 13A-13E illustrate an exemplary cephalomedullary nail lag screw 1300. The screw 1300 can include a hybrid functional gradient lattice structure with the least density of the screw 1300 towards the tip 1302 and the highest density towards the head 1306, as well as less dense lattice along an inferior aspect 1314 (in the orientation as shown in FIGS. 13 and 13A-13D) of the screw 1300. The hybrid functionally graded lattice can incorporate two distinct lattices with variable density designed to withstand the difference in loading of the screw 1300 during in vivo activity/use. The two distinct lattices of the core 1304 can be separated by a divider wall 1311 extending along the length of the core 1304. The divider wall 1311 can run obliquely relative to the longitudinal axis of the screw 1300 to allow for varying proportion of each lattice along the longitudinal axis of the screw. The inferior aspect 1314 of the screw 1300 can withstand a greater compression force relatively, while the superior aspect 1316 (in the orientation as shown in FIGS. 13 and 13A-13D) of the screw 1300 can withstand a greater tensile force relatively. Therefore, the hybrid gradient of the screw 1300 can withstand these different forces placed on the screw. Additionally, the screw 1330 can include a bidirectional cancellous screw gradient with densest portions towards the center of the core 1304 and towards the head 1306 of the screw 1300, which can enhance osscointegration and mitigate stress shielding and cut-out of the screw 1300.

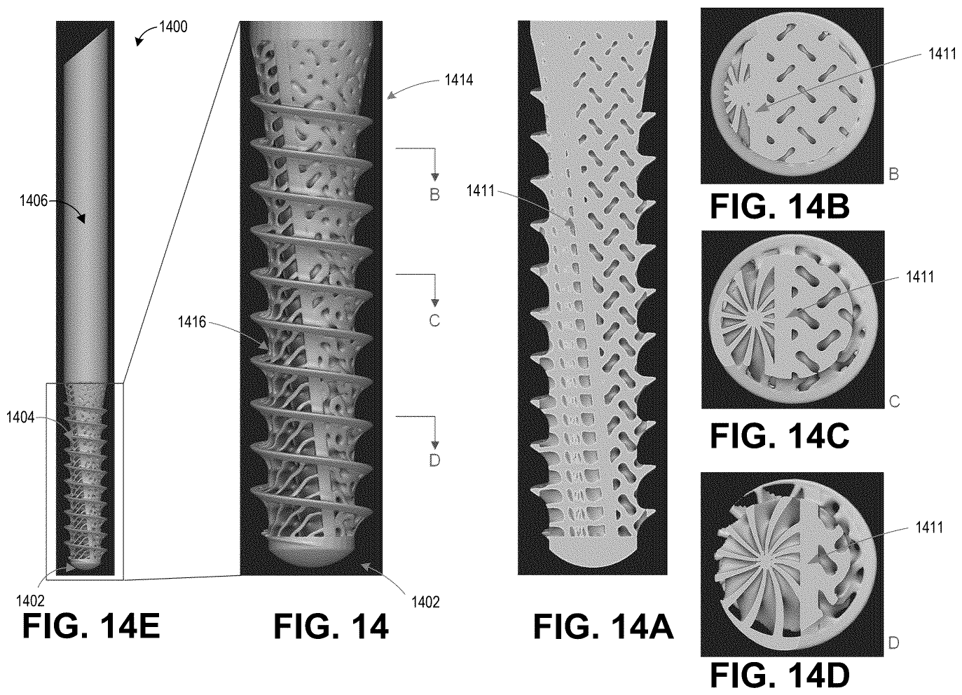

FIGS. 14 and 14A-14E illustrate an exemplary cephalomedullary nail lag screw 1400. The screw 1400 can include a hybrid functional gradient lattice structure with the least density of the screw 1400 at the tip 1402 and the highest density towards the head 1406, as well as less dense lattice along a superior aspect 1416 (in the orientation as shown in FIGS. 14 and 14A-14D) of the screw 1400. The hybrid functionally graded lattice can incorporate two distinct lattices with variable density designed to match the regional bone density of the proximal femur. The two distinct lattices of the core 1404 can be separated by a divider wall 1411 extending along the length of the core 1404. The divider wall 1411 can run obliquely relative to the longitudinal axis of the screw 1400. The inferior aspect 1414 of the screw 1400 can be placed along the medial calcar of the proximal femur, which contains the densest bone in this region. Therefore, the inferior aspect 1414 (in the orientation as shown in FIGS. 14 and 14A-14D) of the screw 1400 can contain a lattice that is more dense to match the surrounding bone. The superior aspect 1416 can be implanted within the less dense cancellous bone and therefore can have a less dense lattice structure to match the surrounding bone. Additionally, the decreased density superiorly can help mitigate cut out and failure of the screw 1400 since a cephalomedullary nail lag screw can fail by cutting out the bone medially and superiorly. Therefore, less density in these areas (towards the tip 1402 and superior aspect 1416 of the screw 1400) can result in less risk of failure. Additionally, the screw 1400 can include a bidirectional cancellous screw gradient with densest portions towards the center of the core 1404 and towards the head 1406 of the screw 1400, which can enhance osscointegration and mitigate stress shielding and cut out of the screw 1400.

The lag screws shown in FIGS. 9, 10, 11, 12, 13, and 14 can incorporate any of the features of one another and/or other lattice embodiments not depicted in the Figures, but may be variants of those lattice embodiments based on the present disclosure. The lag screws can incorporate any of the features of the other types of implants disclosed herein and the other types of implants disclosed herein can incorporate any of the features of the lag screws.

The lag screws disclosed herein can be used for other types of fracture fixation applications, such as percutaneous screw fixation of femoral neck fracture or proximal humerus fracture fixation (described in more details below). The hybrid pattern disclosed herein can vary in any one or more of: types and proportions of lattice, quantities of lattices, shape, size, and/or direction of dividing wall, and/or the like. The hybrid pattern disclosed herein can be applied to proximal humerus fracture fixation, in which the lag screws disclosed herein can be inserted through a plate or through an intramedullary nail. Proximal humerus fractures can occur in osteopenic bone and fixation, with most common complication of osteosynthesis being intra-articular screw penetration through the humeral head. The hybrid lattice pattern disclosed herein can mitigate screw failure.

The screws disclosed herein can be used for percutaneous screw fixation of femoral neck fracture. A number of (for example, two, three, four, or otherwise) of the screws disclosed herein can form a femoral neck fracture fixation kit. The screws in the kit can be cannulated for percutaneous application, where in the screws can be inserted via guidewires.

The screws disclosed herein can be used in plate osteosynthesis for fixing proximal humerus fractures, for example, in a minimally invasive procedure. A number (for example, twelve, sixteen, or otherwise) screws disclosed herein and a locking plate (for example but not limited to the PHILOS plate (Synthes GmbH, Oberdorf, Switzerland)) can form a plate osteosynthesis kit. The locking plate can include a number of holes or apertures at different locations of the plate. The screws may include different diameters, lengths, and/or thread dimensions. For example, some of the bone screws in the kit can function as locking screws and some of the bone screws in the kit can function as cortex screws.

### Example Spinal Fixation Application

In some implementations, the orthopedic fixation implant disclosed herein can be used in spinal fixation. As a non-limiting example, the orthopedic fixation implant disclosed herein can serve as a pedicle screw. An example spinal fixation kit may include a plurality of the orthopedic fixation implants disclosed herein, each serving as a pedicle screw or sacropelvic fixation screw. The kit may include a rod. Each pedicle screw or sacropelvic fixation screw may couple with a rod coupling head fastened to the rod to adjoin adjacent spinal segments.

### Example Reverse Shoulder Arthroplasty Application

In certain orthopedic surgical techniques, an implant can be stabilized with a bone screw (threaded into the bone), or a cylindrical post without threads (impacted via press-fit technique into the bone) as standard of care. With a screw (such as screws with a screw head, a variable thread pitch, and/or otherwise), one can compress the implant (for example, an implant metal plate) into the bone, thus providing continuous compression for improved initial stability. However, traditional screws may not be as good as a post with certain amount of porosity for encouraging bone ingrowth and optimizing mechanical strength properties to match the region of bone at insertion. Osseointegration can be desirable for many orthopedic devices and implants. The screws disclosed herein combines the advantages of both a screw and a post by both improving stability and promoting osseointegration.

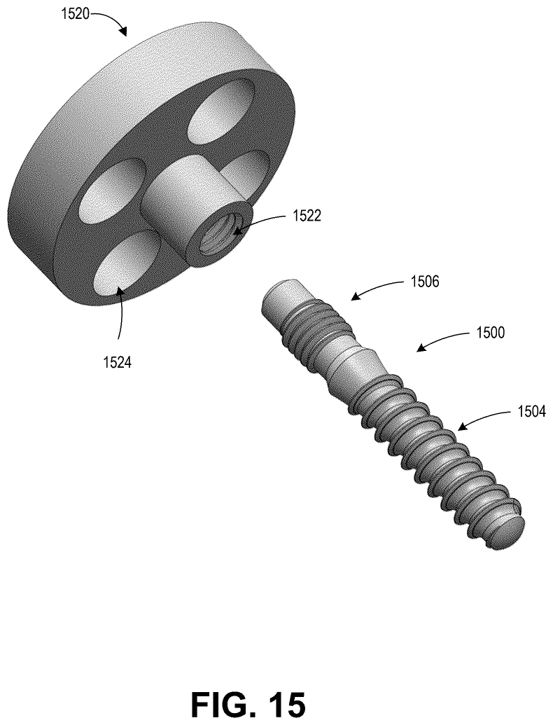

FIG. 15 illustrates a reverse shoulder baseplate 1520 in combination with a central screw 1500, which can incorporate any of the features of the bone screws or lag screws disclosed herein. The baseplate 1520 and the screw 1500 can be part of a reverse shoulder arthroplasty kit. The baseplate 1520 can be placed onto the glenoid during a reverse shoulder replacement surgery. The head 1506 of the central screw 1500 can threadedly or otherwise engage the baseplate 1520. In the implementation as shown, threads on the head 1506 of the central screw 1500 can engage internal threads on a central bore 1522 of the baseplate 1520, and threads on the core 1504 of the screw 1500 can be implanted into bone to secure the baseplate 1520 onto the glenoid. In some implementations, the orthopedic fixation implant examples disclosed herein can additionally or alternatively serve as a peripheral screw that can be placed into one of the peripheral holes 1524 (which may optionally be threaded) of the baseplate 1520 to further secure the baseplate 1520 at the glenoid. The screws shown in FIGS. 15, 16, 17, and 18 can incorporate any of the features of one another. The reverse shoulder arthroplasty screws can incorporate any of the features of the other types of implants disclosed herein and the other types of implants disclosed herein can incorporate any of the features of the reverse shoulder arthroplasty screws.

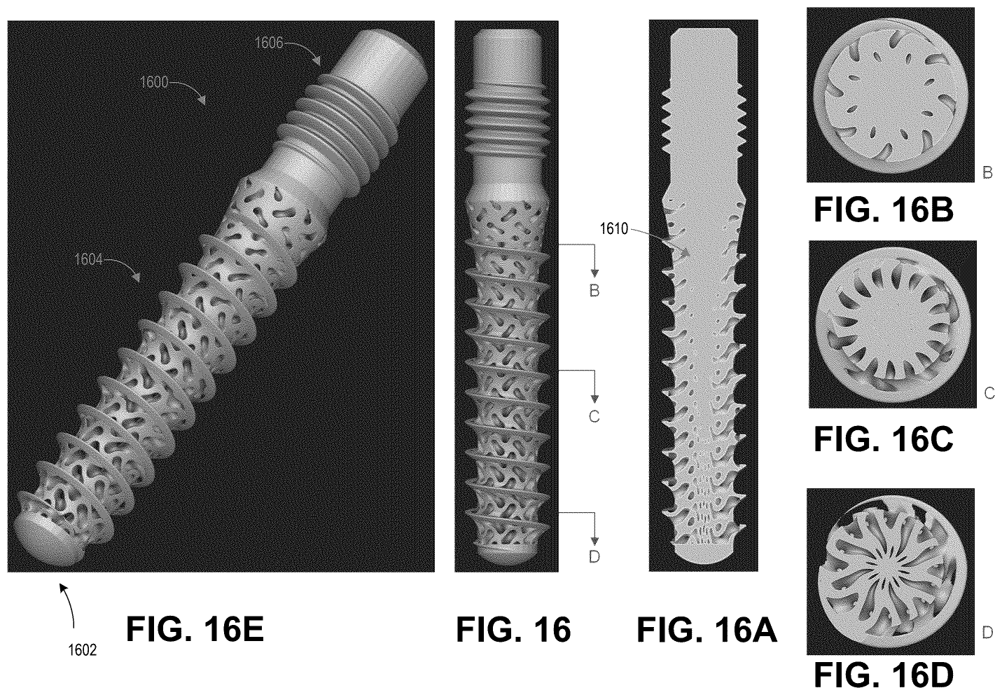

FIGS. 16 and 16A-16E illustrate an exemplary screw 1600. The screw 1660 can serve as a central screw for fixing any suitable reverse shoulder baseplate to the glenoid. The core 1604 of the screw 1660 can include functionally graded circular gyroid structure, with the lowest density of lattice toward the tip 1602 (see FIG. 16D) and the periphery of the core 1604 (see FIG. 16A), and the highest density towards the head 1606 (see FIG. 16B) and the center of the core 1604 (see FIG. 16A). The solid inner core 1610 of the screw 1600 can taper down toward the tip 1602.

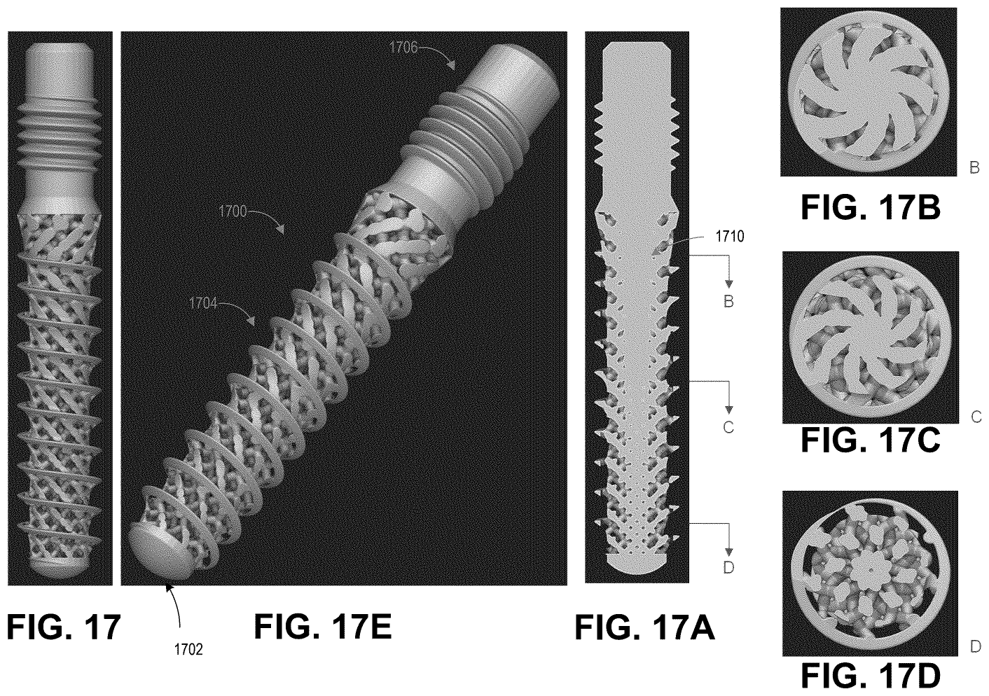

FIGS. 17 and 17A-17E illustrate an exemplary screw 1700. The screw 1700 can serve as a reverse shoulder arthroplasty central screw. The screw 1700 can include functionally graded circular graphed diamond structure, with the lowest density of lattice toward the tip 1702 (see FIG. 17D) and the periphery of the core 1704 (see FIG. 17A), and the highest density towards the head 1706 (see FIG. 17B) and the center of the core 1704 (see FIG. 17A). The inner core 1710 of the screw 1700 can taper down towards the tip 1702.

In reverse shoulder arthroplasty, there are certain benefits in using screw fixation and certain benefits for using post fixation with ingrowth features as described elsewhere in the present disclosure. The screw 1600, 1700 combines the benefits of the screw for compression of the baseplate into the bone and optimization of the core of the screw for ingrowth of bone and enduring fixation of the baseplate. Additionally, the functional graded nature of the lattice can minimize stress shielding around the reverse shoulder arthroplasty baseplate and screw. Stress shielding can lead to glenoid component loosening in the long term in existing reverse shoulder arthroplasty implants. Another feared complication of reverse shoulder arthroplasty is the complication of scapula spine fractures, which are commonly thought to be due to a stress riser that forms at the tip of the screw used to fix the baseplate. The tip 1602, 1702 of the screw 1600, 1700 being less dense due to the less dense lattice in the distal portion of the core 1604, 1704 can better match the modulus of elasticity of the surrounding bone, therefore mitigating the risk of a stress riser and a stress fracture emanating from the tip 1602, 1702 of the screw 1600, 1700.

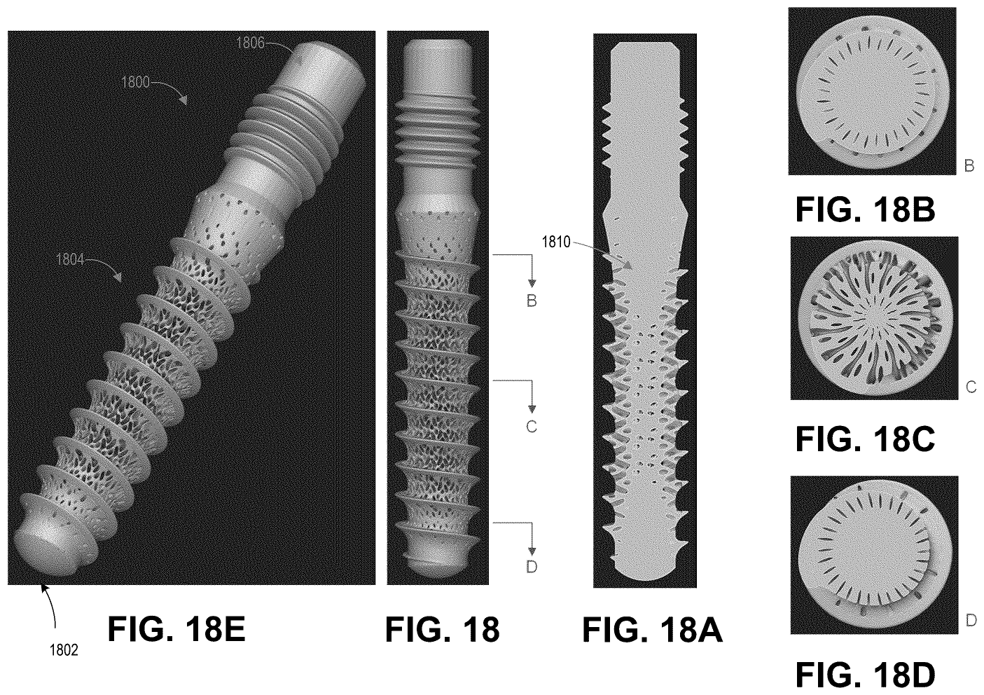

FIGS. 18 and 18A-18E illustrate exemplary screw 1800. The screw 1800 can serve as a reverse shoulder arthroplasty central screw. The screw 1800 can include functionally graded circular splitP structures, with a highest density towards the head 1806 (FIG. 18B) and the tip 1802 (FIG. 18D) of the screw 1800 and towards the center of the core 1804, and the least density towards the mid-portion (between the head 1806 and the tip 1802, see, e.g., FIG. 18C) of the screw 1800 as well as the periphery of core 1804. As shown in FIG. 18A, the inner core 1810 can include an hourglass gradient.

The screw 1800 can have any of the technical and/or mechanical advantages as the screw 1600, 1700, except that the screw 1800 can serve as a cortical screw with its cortical style longitudinal gradient. The screw 1800 can have greater density at both the head 1806 and the tip 1802 of the screw 1800. In reverse shoulder arthroplasty, for screws that are placed bicortically through a baseplate, the screw 1800 can provide a more similar (that is, better matching of) the modulus of elasticity with the surrounding bones, with the tip 1802 and head 1806 being within cortical bone and the central portion (between the tip 1802 and the head 1806) being within cancellous bone. The hourglass gradient may advantageously aid in screw insertion as well as improving the stress dissipation between the screw tip and the core to prevent screw breakage.

Similar to reverse shoulder arthroplasty, the screws 1600, 1700, 1800 can be used in hip arthroplasty for stabilizing an acetabular cup. The screws disclosed herein and the acetabular cup can form a hip joint arthroplasty kit.

### Example Anchors

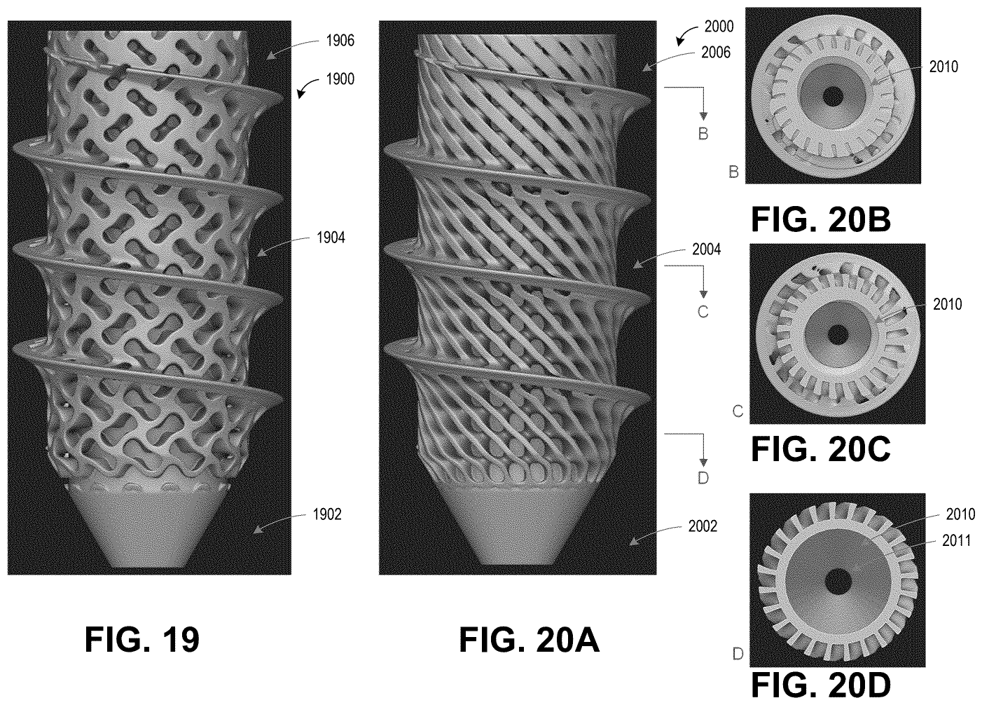

FIG. 19 illustrates a functionally graded gyroid lattice bone anchor 1900. The bone anchor 1900 can have a head 1906 and a core 1904 that both include the lattice structures (which may or may not be the same lattice structure). Externally the head 1906 and the core 1904 can be one continuous surface and therefore contain one continuous lattice. Internally the head 1906 contains the seating for a driver to advance the implant forward and the core 1904 contains the mating threads for insertion of a fastening component. The anchor 1900 can have a solid tip 1902 (i.e., without a lattice structure but may still include cannulation). The bone anchor 1900 can have the highest density of lattice towards the head 1906 of the anchor 1900 and the lowest density towards the tip 1902 of the anchor 1900.

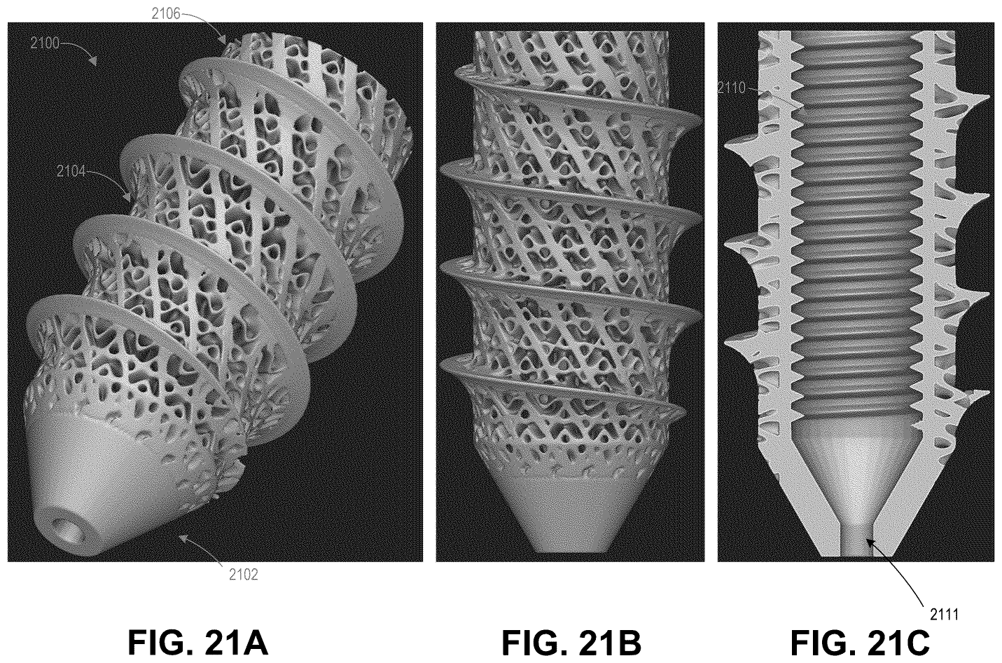

FIGS. 20A-20D illustrate a functionally graded diamond lattice bone anchor 2000. The bone anchor 2000 can have a head 2006 and a core 2004 that both include the lattice structures (which may or may not be the same lattice structure). The anchor 2000 can have a solid tip 2002 (i.e., without a lattice structure but may still include cannulation). The bone anchor 2000 can have the highest density of lattice towards the head 2006 of the anchor 2000 and the lowest density towards the tip 2002 of the anchor 2000. FIGS. 20B-20D represent cross sections at proximal, mid portion (between the proximal and distal portions), and distal locations of the bone anchor 2000, demonstrating a decreased wall thickness and increased pore 2010 size towards the tip 2002 of the bone anchor 2000. The bone anchor 2000 can further include a throughbore or channel 2011. The throughbore or channel 2011 can be used for passing a suture and/or a guidewire.

Bone anchors can be used for fixation of tendon to bone in the setting of torn tendons, such as rotator cuff tears or quadriceps tendon tears. The bone anchor 1900, 2000 can include a longitudinal cancellous screw style gradient with the least density towards the tip 1902, 2002 of the anchor 1900, 2000 since these anchors are placed unicortically through one layer of cortex and into cancellous bone. The functional grading of the bone anchor 1900, 2000 can better match in stiffness to the surrounding bone, enhance osscointegration, and/or mitigate stress shielding. Additionally, having a lattice structure within a superior aspect of the screw (for example, in the head 1096, 2006) can allow tendon to grow into the lattice as well as continuously into the surrounding bone.

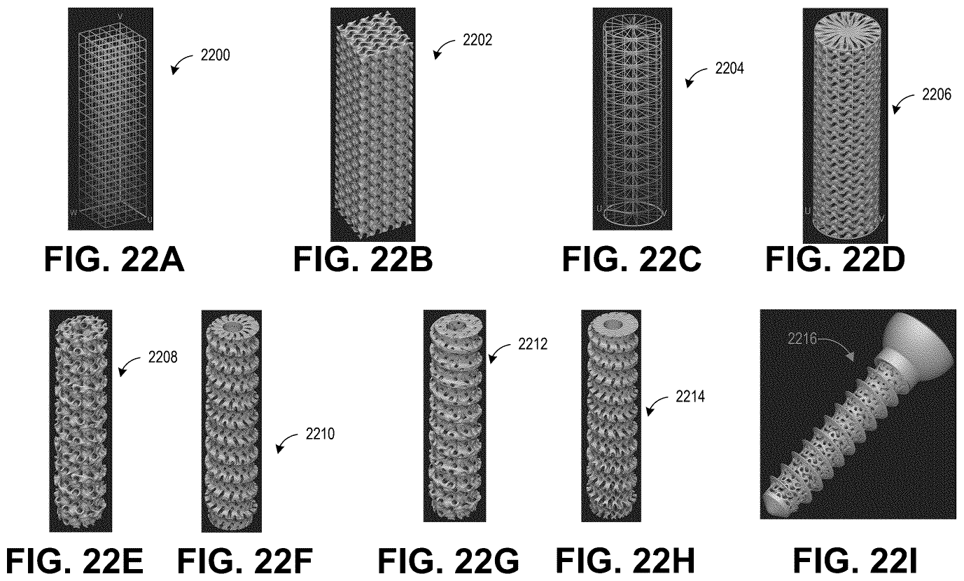

FIGS. 21A-21C illustrate an exemplary bone anchor 2100. The bone anchor 2100 can include a bore 2110 of a relatively uniform size in the head 2106 and core 2104. The bore 2110 can include internal threads. The bone anchor 2100 can further include a throughbore or channel 2111. The throughbore or channel 2111 can be used for passing a suture and/or a guidewire. The tip 2102 of the bone anchor 2100 can be solid (i.e., without a lattice structure but may still include cannulation). The bone anchor 2100 can include a superimposed or interpenetrating lattice combination propagated through the head 2106 and the core 2104 of the bone anchor 2100. For example, the combination of lattice can include diamond and splitP, or other suitable combinations. The superimposed lattice combination can include an interpenetrating lattice. The superimposed or interpenetrating lattice combination can enhance the mechanical strength and structural properties compared to individual lattice patterns.

The suture anchors shown in FIGS. 19, 20A, and 21A can incorporate any of the features of one another. The suture anchors can incorporate any of the features of the other types of implants disclosed herein and the other types of implants disclosed herein can incorporate any of the features of the suture anchors. In some implementations, a rotator cuff repair kit can include a number (for example, two, four, six, eight, or otherwise) of the suture anchors disclosed herein.

### Example Features for Removal of Orthopedic Fixation Implant

In some implementations, additional implant components (for example but not limited to a metal plate, an intramedullary nail, a reverse shoulder baseplate, etc.) coupled with the orthopedic fixation implants disclosed herein can have features that may allow for and/or facilitate removal of the orthopedic fixation implant. In some embodiments, the additional implant component may include a number of slots surrounding an aperture for receiving an orthopedic fixation implant. In some embodiments, the additional implant component may alternatively include a number of holes surrounding an aperture for receiving an orthopedic fixation implant. In some embodiments, the slots may be straight or curved. In some embodiments, the slots may allow for osteotome advancement alongside the bone-implant interface. In some embodiments, the holes may allow for a wire or drill bit to advance alongside the bone-implant interface.

### Example Method of Creating Lattice in Orthopedic Fixation Implant

The 3D printed orthopedic fixation implant with functional gradient lattice can be created first by selecting a unit cell, or the TPMS or lattice geometry to be patterned in a solid body. The triply periodic, minimal surface structures can include a diamond unit cell in FIG. 2A, a gyroid unit cell in FIG. 2B, a lidinoid unit cell in FIG. 2C, a splitP unit cell in FIG. 2D, a diamond unit cell, or other suitable shapes.

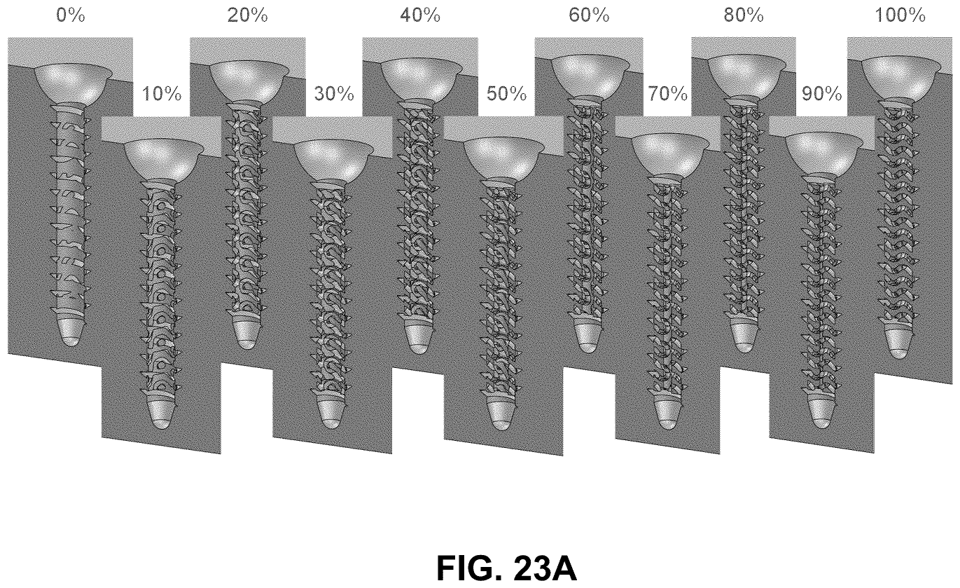

With the selected geometry, a cell map can be generated by creating a distribution of cell sizing and spacing within which each unit cell can be patterned and interconnected across the map to create a full continuous structure, such as rectangular gyroid structures 2200, 2202 in FIGS. 22A and 22B and cylindrical gyroid structures 2204, 2206 in FIGS. 22C and 22D. The cell map can determine a density of the lattice, with variables, such as a cell size (which can be in polar and/or cartesian coordinates) within which the unit cell fits, and/or a wall thickness, which can determine a pore size of the lattice.

Intersections can next be performed between a solid body and a mapped TPMS structure. FIGS. 22E and 22F illustrate the example resulting intersections, such as a rectangular gyroid body 2208 and a cylindrical gyroid body 2210. The solid body overlapping with the propagated TPMS-mapped structure can be combined with a Boolean intersection operation to keep only the geometry in contact between the solid and the lattice.

Grading of the lattice can next be added to the TPMS body covering a prescribed range of wall thickness. FIG. 22G illustrates grading added to the rectangular gyroid body 2212. FIG. 22H illustrates grading added to the cylindrical gyroid body 2214. From a starting feature, such as a plane or an object, a scalar field can be applied with a minimum and maximum thickness that increments up to a maximum distance from the starting point. The resulting gradient wall thickness can directly correspond to the pore/opening size of the lattice.

FIG. 22I illustrates a complete combined body of a graded TPMS implant 2216. All additional features can be added to a Boolean union operation where all bodies can be merged into a single object. Any of the orthopedic fixation implant examples disclosed herein can be created using this method.

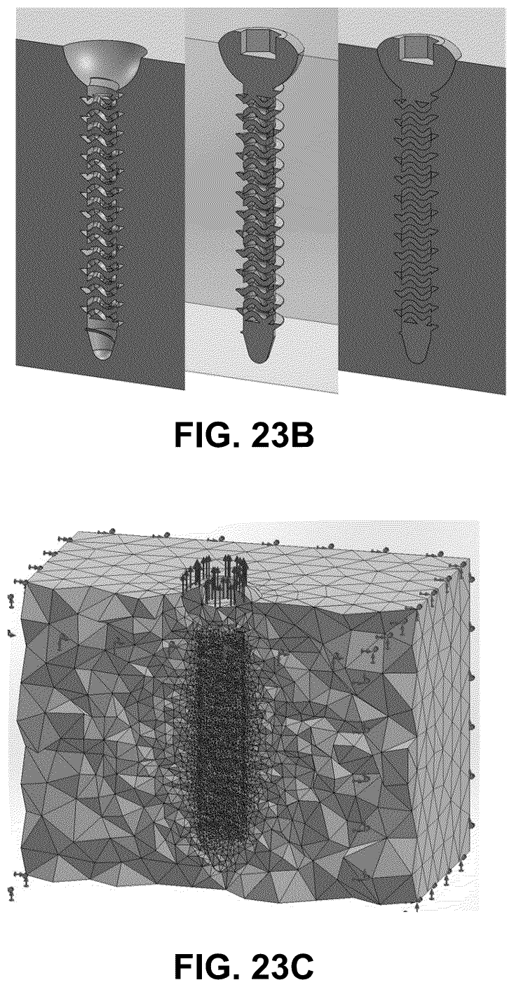

FIGS. 23A-23C illustrate bone ingrowth simulation of an example orthopedic fixation implant with functional grading lattice in the core. FIG. 23A illustrates simulated increased ingrowth percentages over time. FIG. 23B illustrates the orthopedic fixation implant at 100% simulated ingrowth. FIG. 23C illustrates simulated pullout performance and the contact density of the implant at 100% ingrowth.

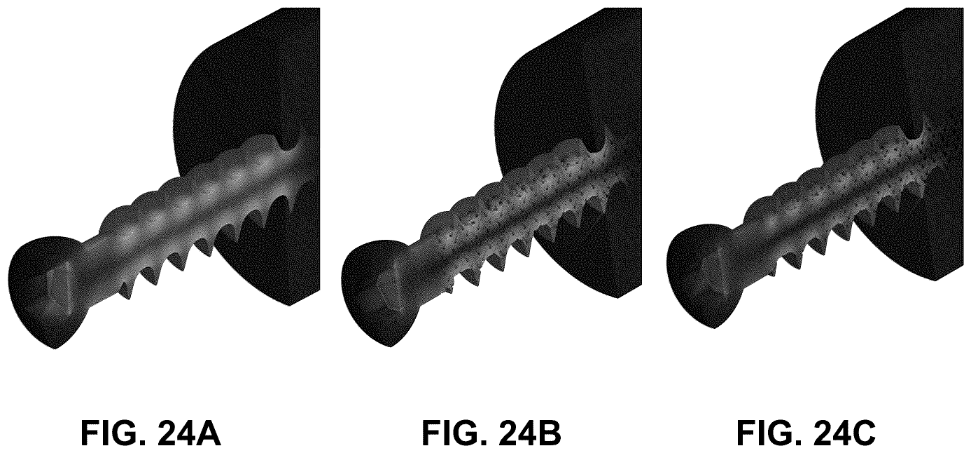

FIGS. 24A-24C, 25A-25C, 26A-26C, and 27A-27B illustrate finite element analysis (FEA) simulations of screw installation in the form of a Von Mises stress distribution.

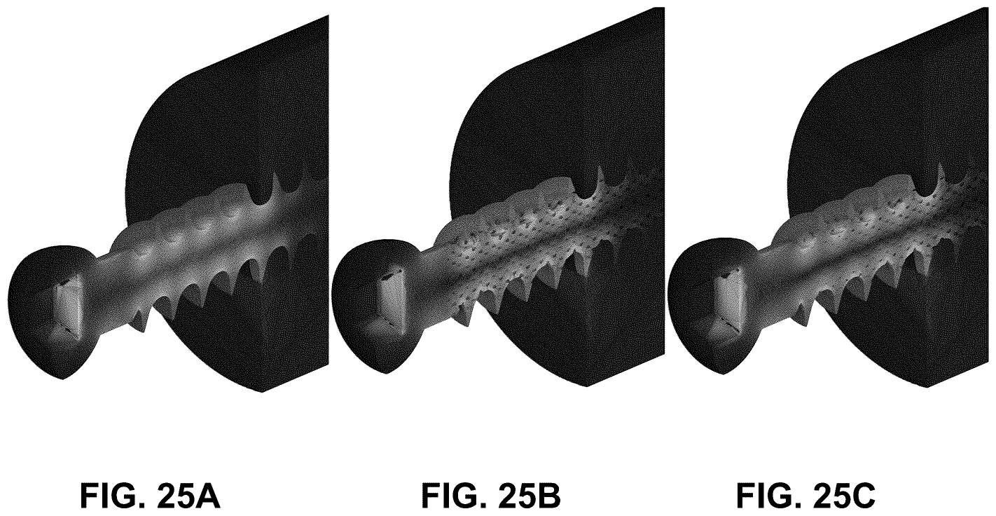

FIGS. 24A-24C compare the impact of torsional load during insertion of a solid titanium screw (24A), a non-graded lattice core screw (24B), and a graded lattice core screw (24C) at a screw insertion depth of 60%. FIGS. 25A-25C compare the impact of torsional load during insertion of a solid titanium screw (25A), a non-graded lattice core screw (25B), and a graded lattice core screw (25C) at a screw insertion depth of 75%. The results shown in FIGS. 24A-24C and 25A-25C demonstrate that, instead of a uniform stress distribution of the solid and non-graded screws, the graded screw design can allow for non-uniform stress dissipation along the longitudinal gradient of the screw, thus minimizing stress at the screw head-shaft junction, which is a typical location of screw breakage/failure under torsional load.

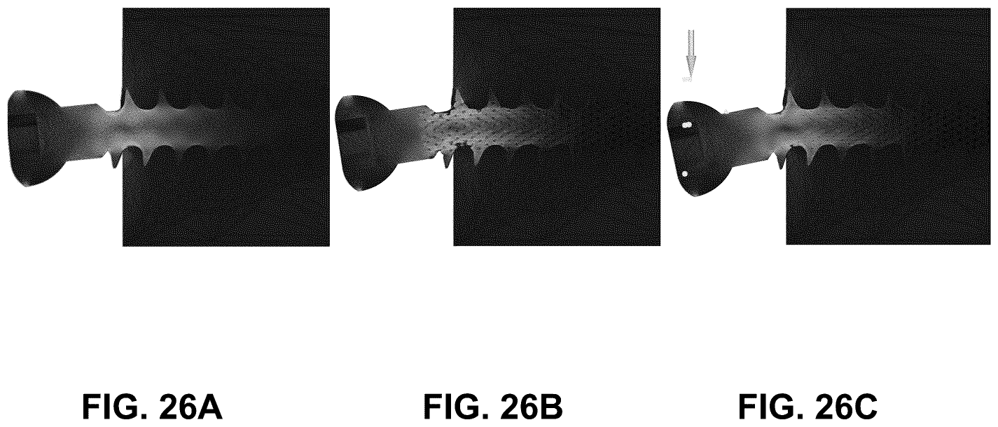

FIGS. 26A-26C compare the impact of bending load as a result of standard bodily motion of a solid titanium screw (26A), a non-graded lattice core screw (26B), and a graded lattice core screw (26C) at a screw insertion depth of 85%. In the illustrated embodiment, a bending load of 220 N may be applied to the head of a screw with a 40 mm shaft length. The results in FIGS. 26A-26C demonstrate that the graded screw design can allow for stress similar in magnitude and location to the solid screw design, whereas a non-graded lattice may have substantially greater stress. Additionally, the graded screw (0.179 mm) in FIG. 26C showed more similar displacement to the solid screw (0.157 mm) in FIG. 26A, when compared to the non-graded screw (0.232 mm) in FIG. 26B. Example load position and direction is reflected in the graded lattice image in FIG. 26C.

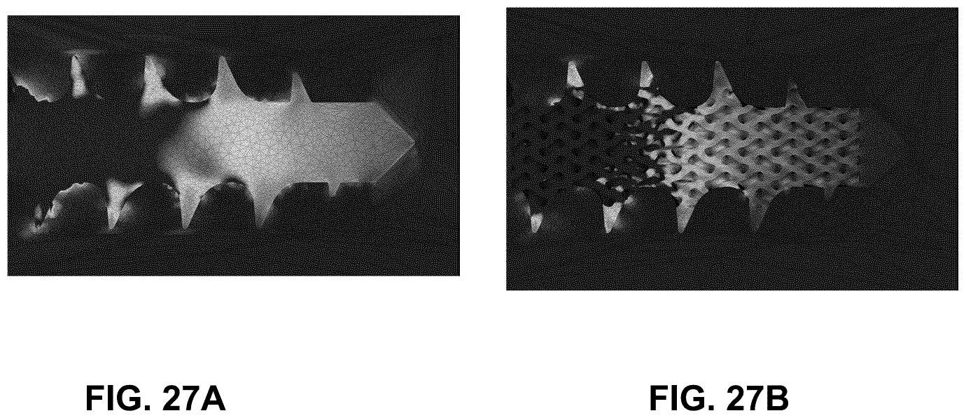

FIGS. 27A and 27B compare FEA simulations of the impact of axial loading at the tip of a fully inserted solid titanium screw (A) and a graded lattice core screw (B). In the illustrated embodiment, an axial loading (1220 N) may be at the tip of a fully inserted screw. The results in FIGS. 27A and 27B demonstrate that the graded screw design can allow for better stress dissipation along the length of the shaft and decrease overall stress when approaching the screw tip compared to the solid screw design.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A three-dimensionally (“3D”) printed orthopedic fixation implant configured to reduce regional implant stress and enhance osseointegration when inserted into bone of a patient, the implant comprising:
a head defining a proximal end of the implant;
a tip defining a distal end of the implant;
a core extending from the head to the tip, the core being elongate; and
threads extending along at least a portion of the core,
wherein one or more functionally graded lattice structures are propagated longitudinally and/or radially in at least portions of the core, a density of the one or more functionally graded lattice structures having a varying longitudinal gradient and a varying radial gradient.

2. The implant of claim 1, wherein the head is a solid head without any lattice structure.

3. The implant of claim 1, wherein the one or more functionally graded lattice structures are propagated further into the head.

4. The implant of claim 3, wherein the implant is a suture anchor configured to secure a tendon or ligament to bone.

5. The implant of claim 1, wherein the tip is a solid tip without any lattice structure.

6. The implant of claim 1, wherein the one or more functionally graded lattice structures are propagated further into the tip.

7. The implant of claim 6, wherein the one or more functionally graded lattice structures are propagated further into the threads.

8. The implant of claim 1, wherein the varying radial gradient density is highest toward a center of the core and lowest toward a periphery of the core.

9. The implant of claim 1, wherein the varying longitudinal gradient density is highest toward the head and lowest toward the tip.

10. The implant of claim 9, wherein the implant is configured to be inserted unicortically with the head surrounded by cortical bone and the tip surrounded by cancellous bone.

11. The implant of claim 1, wherein the varying longitudinal gradient is highest toward the head and the tip, and lowest at a location along the core between the head and the tip.

12. The implant of claim 11, wherein the implant is configured to be inserted bicortically with the head and the tip surrounded by cortical bone and a mid-portion of the core surrounded by cancellous bone.

13. The implant of claim 1, wherein an outer shell of the core is solid.

14. The implant of claim 1, wherein the one or more functionally graded lattice structures extend through an outer surface of the core.

15. The implant of claim 1, wherein the core comprises a solid inner core.

16. The implant of claim 1, wherein the one or more functionally graded lattice structures comprise triply periodic minimal surface (TPMS) lattices.

17. The implant of claim 16, wherein the TPMS lattices comprise one or more of gyroid, diamond, lidinoid, or splitP geometries.

18. The implant of claim 1, wherein the one or more functionally graded lattice structures comprise a combination of different lattice geometries.

19. The implant of claim 1, wherein the one or more functionally graded lattice structures comprise two or more functionally graded lattice structures that are interpenetrating or overlapping.

20. The implant of claim 1, wherein the one or more functionally graded lattice structures comprise auxetic lattice structures.
